(19) United States
(12) Patent Application Publication
(10) Pub. No.: US 2003/0138449 A1
(43) Pub. Date: Jul. 24, 2003

(54) Method of administering FimH protein as a vaccine for urinary tract infections

(75) Inventors: Solomon Langermann, Baltimore, MD (US); W. Ripley Ballou JR., Silver Springs, MD (US)

Correspondence Address: PENNIE AND EDMONDS, 1155 AVENUE OF THE AMERICAS, NEW YORK, NY, 100362711

(73) Assignee: MedImmune, Inc.

(21) Appl. No.: 10/306,897

(22) Filed: Nov. 27, 2002

Related U.S. Application Data
(63) Continuation of application No. 09/724,397, filed on Nov. 28, 2000, now abandoned.
(60) Provisional application No. 60/226,146, filed on Aug. 18, 2000.

(51) Int. Cl. A61K 39/02

(52) U.S. Cl. 424/190.1

(57) ABSTRACT

The present invention relates to methods of stimulating an immune response in a primate utilizing compositions comprising bacterial adhesin proteins and/or immunogenic fragments thereof. The compositions are useful for the prevention and treatment of bacterial induced diseases involving bacterial adherence to a target cell, such as diseases of the urinary tract. More specifically, the invention relates to the vaccination of primates, preferably humans, with protein complexes, such as a purified FimH polypeptides, a purified FimC-FimH (FimCH) polypeptide complex, or immunogenic fragments thereof, to stimulate protective immunity in the recipient against infection by pathogenic bacteria, including all types of Enterobacteriaceae, preferably E. coli to produce specific immunoglobin molecules in the serum and urine or mucosal secretions of the subject.

This application claims the benefit of priority to U.S. Patent Application Serial No. 60/226,146, filed Aug. 18, 2000, which is incorporated herein in its entirety.

### 1. INTRODUCTION

The present invention relates to methods of stimulating an immune response in a primate utilizing compositions comprising bacterial adhesin proteins and/or immunogenic fragments thereof. The compositions are useful for the prevention and treatment of bacterial induced diseases involving bacterial adherence to a target cell, such as diseases of the urinary tract. More specifically, the invention relates to the vaccination of primates, preferably humans, with adhesin protein complexes, such as a purified FimH polypeptides complexes, purified FimC-FimH (FimCH) polypeptide complexes, or immunogenic fragments thereof, to stimulate protective immunity in the recipient against infection by pathogenic bacteria, including all types of Enterobacteriaceae, preferably E. coli.

### 2. BACKGROUND OF THE INVENTION

Urinary tract infections (herein, “UTI”) present a disease process that is mediated (or assisted or otherwise induced) by the attachment of bacteria to cells. Escherichia coli (E. coli) is the most common pathogen of the urinary tract, accounting for more than 85% of cases of asymptomatic bacteriuria, acute cystitis and acute pyelonephritis, as well as greater than 60% of recurrent cystitis, and at least 35% of recurrent pyelonephritis infections. Furthermore, approximately 25%-30% of women experience a recurrent E. coli urinary tract infection within the first 12 months following an initial infection but after a second or third infection the rate of recurrence increases to 60%-75%. Given the high incidence, continued persistence, and significant expense associated with E. coli urinary tract infections, there is a need for a prophylactic vaccine to reduce susceptibility to this disease.

To initiate infection, bacterial pathogens must first be able to colonize an appropriate target tissue of the host. For many pathogens this tissue is located at a mucosal surface, in particular in the urogenital tract with respect to urinary tract infections.

Colonization begins with the attachment of the bacterium to receptors expressed by cells forming the lining of the mucosa. Attachment is mediated via proteins on the bacterium that bind specifically to the target cell via a cellular receptor of some kind, nonlimiting examples of receptors include naturally occurring transmembrane receptors or carbohydrate moieties. These bacterial proteins, or adhesins, are expressed either directly on the surface of the bacterium, or more typically, as components of elongated rod-like protein structures called pili, fimbriae or fibrillae.

While many factors contribute to the acquisition and progression of E. coli urinary tract infections, it is generally accepted that colonization of the urinary epithelium is a required step in the infection process. In a typical course of E. coli urinary tract infection, bacteria originate from the bowel, ascend into the bladder, and adhere to the bladder mucosa where they multiply and establish an infection (cystitis) before ascending into the ureter and kidney. Thus, disruption or prevention of pilus-mediated attachment of E. coli to urinary tract epithelial cells may prevent or retard the development of urinary tract infections. In this regard, a number of studies have pointed to a role for pili in mediating attachment to host bladder mucosal cells.

The bladder mucosa is comprised of layers of cells starting with the luminal surface and is lined with stratified transitional epithelium (“urothelium”) which is usually three to four cell layers thick. A thin basement membrane and lamina propria separate the epithelial cells from the smooth muscular and serous layers of the outer wall of the bladder. The urothelium is comprised of small and relatively undifferentiated basal and intermediate epithelial cells underlying a single layer of highly differentiated, large, multinucleate superficial facet cells expressing integral membrane glycoproteins. The glycoproteins serve as points of attachment or adherence by invading pathogens.

Type 1 pili are thought to be important in initiating colonization of the bladder and inducing cystitis, whereas P pili are thought to play a role in ascending infections and the ensuing pyelonephritis. Such pili are heteropolymeric structures that are composed of several different structural proteins required for pilus assembly. P pili-carrying bacteria recognize and bind to the gal-(α1-4)gal moiety present in the globoseries of glycolipids on kidney cells in mammals. Type 1 pili-carrying bacteria recognize and bind to D-mannose in glycolipids and glycoproteins of the urothelium.

PapG, the adhesin protein in P pili bacteria that mediates the specific interaction of the pilus with receptors on the surface of host cells, is found at the distal end of the tip fibrillum. Its periplasmic chaperone protein is PapD which is highly conserved across strains of E. coli. (Hultgren et al., Proc. Natl. Acad. Sci. USA 86:4357 (1989); Hung et al., EMBO Journal 15:3792-3805 (1996).

With regard to type 1 pili, tip adhesins and other ancillary subunits also have been identified. The FimH polypeptide is the D-mannose-binding adhesin that promotes attachment of type 1 piliated bacteria to host cells via mannose-containing glycoproteins on eukaryotic cell surfaces. FimC is its periplasmic chaperone protein. The FimH polypeptide is also highly conserved not only among uropathogenic strains of E. coli, but also among a wide range of gram-negative bacteria. For example, all Enterobacteriaceae produce FimH, thus, vaccines incorporating FimH should exhibit a broad spectrum of protection.

It has recently been reported that such chaperones can direct formation of the appropriate native structure of the corresponding adhesin or pilin by inserting a specific fold of the chaperone protein in place of a missing domain or helical strand of the chaperone or pilin. Thus, FimH proteins tend to have their native structure in the presence of such a chaperone but not in its absence (Choudhury et al., X-ray Structure of the FimC-FimH Chaperone-Adhesin Complex from Uropathogenic E. coli, Science 285:1061 (1999); Sauer et al., Structural Basis of Chaperone Function and Pilus Biogenesis, Science 285:1058 (1999)). In addition, recent publications have indicated that the required chaperone strand can be inserted into the adhesin or pilin protein, such as FimH, to provide the missing structure and produce the correct native structure.

Vaccination techniques have been developed wherein the vaccine composition is delivered to the subject directly at mucosal tissues, such as gut associated lymphoid tissue (GALT), nasopharyngeal lymphoid tissue (NALT) and bronchial-associated lymphoid tissue (BALT), thereby providing localized immunity. Mucosal humoral immunity has been generally thought to come from the secreted form of immunoglobulin, IgA. However, to date, there are no reports of systemic administration of a FimH vaccine composition to a primate which stimulates a humoral immune response sufficient to provide protective immunity at mucosal tissues in humans, with respect to urogenital tract infections.

While other antigens have been utilized to produce antibodies for diagnosis and for the prophylaxis and/or treatment of bacterial urinary tract infections, there is a need for improved or more efficient vaccines for use in primates, and more particularly in humans. Such vaccines should have an improved or enhanced effect in preventing bacterial infections mediated by adhesins and pili sufficient to prevent or treat UTI in humans.

### 3. SUMMARY OF THE INVENTION

The present invention is based, in part, upon the surprising discovery that non-mucosal administration to a primate of a FimCH complex resulted in the presence of IgG molecules specific for FimCH in the genital secretions of the primate, the presence of which IgG molecules correlated with a reduction in the incidence of urogenital tract infections.

Accordingly, the present invention relates to methods of stimulating an immune response in a primate utilizing purified bacterial adhesin proteins and/or antigenic or immunogenic fragments thereof, preferably fragments containing the attachment domain of the adhesin protein. Compositions comprising the bacterial adhesin proteins or antigenic or immunogenic fragments thereof are useful for the prevention and treatment of bacterial induced diseases involving bacterial adherence to a target cell, such as diseases of the urinary tract.

More specifically, the invention relates to the vaccination of primates, preferably humans, with adhesin proteins or protein complexes thereof, such as purified FimCH proteins, or immunogenic fragments thereof, that stimulate protective immunity against infection by pathogenic bacteria, including types of Enterobacteriaceae, and particularly including type 1 pilin containing gram negative bacteria, e.g., E. coli. These methods result in prophylactic or therapeutic levels of immunoglobulins, particularly, IgGs specific for the adhesin protein in the urine or genital secretions of the recipient. Preferably the IgGs specific for the adhesin protein, preferably FimH, inhibit binding of the bacteria to cell surface residues, for example, inhibit the binding of E. coli to mannose residues, particularly mannose residues on urogenital tract epithelial cell walls, and thus prevent or reduce attachment of E. coli to cells of the bladder, kidney and urinary tract.

The present invention encompasses a method of inducing immunoglobulin molecules, that specifically bind a type 1 pilin polypeptide (or any antigenic or immunogenic fragment thereof, preferably, the attachment domain) associated with a bacterium that causes urogenital tract infections, in the urine or genital tract secretions of a primate. The method comprises administering to a primate a purified peptide or peptide complex comprising a type 1 pilin polypeptide or antigenic or immunogenic fragment thereof (e.g., the attachment domain), which administration induces the presence of immunoglobulin molecules in the urine or genital tract secretions of the primate sufficient to reduce the incidence of urogenital tract infections. Such method also leads to levels of such immunoglobulin molecules in the serum of the primate sufficient to result in protective levels of adhesin protein-specific immunoglobulins in the urine and/or genital secretions of the primate. Additionally, the method also leads to the increase or presence in the serum and/or mucosal secretions of an activity that inhibits binding of the bacterium to cell surface molecules.

The present invention also encompasses a method for eliciting an immune response to a type 1 pilin polypeptide (preferably the attachment domain) associated with a bacterium that causes urogenital tract infection in a primate, which method comprises administering to a primate in need thereof, a purified peptide or peptide complex comprising a type 1 pilin polypeptide (e.g., the attachment domain) in an amount effective to produce immunoglobulin molecules that specifically bind the type 1 pilin polypeptide in serum and in the urine or genital tract secretions of the primate, the level of the immunoglobulin molecules in the serum and, preferably, in the mucosal secretions, being sufficient to reduce the incidence of the urogenital tract infection.

Additionally, in other embodiments, the present invention provides a method for vaccinating a primate against urogenital tract infection, which method comprises administering to the primate, a purified peptide or peptide complex comprising a bacterial type 1 pilin polypeptide (or antigenic or immunogenic fragment thereof, e.g., the attachment domain) associated with a bacterium that causes a urogenital tract infection, in an amount effective to produce immunoglobulin molecules that specifically bind the type 1 polypeptide.

In a specific embodiment, the present invention encompasses a method for preventing or slowing the progression of a urinary tract infection into end stage renal disease in a primate in need thereof, which method comprises administering to the primate a purified peptide or peptide complex comprising a bacterial type 1 polypeptide (or any immunogenic or antigenic fragment thereof, for example, an attachment domain fragment), associated with a bacterium that causes a urogenital tract infection, in an amount effective to produce immunoglobulin molecules that specifically bind the type 1 pilin polypeptide.

The present invention further provides methods for treating or ameliorating the symptoms of a urogenital tract infection in a primate, by administering an adhesin protein of the invention associated with a bacterium that causes a urogenital tract infection in an amount effective to produce IgG molecules that specifically binds the protein.

Also encompassed by the invention are kits and pharmaceutical compositions for use in the methods disclosed herein.

The present invention encompasses methods of prophylaxis for the prevention of urogenital tract infections, preferably urinary tract infections, using the vaccine compositions disclosed herein, particularly in subjects at high risk of such infections, including but not limited to subjects who have already had more than one or two or three UTIs per year, pregnant subjects, subjects with asymptomatic bactourea, particularly pregnant women with reduced levels of IL-6 and/or IL-8 and diabetics, subjects with a familial susceptibility to UTI, subjects with end stage renal disease, subjects with infectious diseases, cancer, HIV and other secondary illnesses, and hospitalized and immunocomprised subjects.

In a preferred embodiment, the FimH compositions of the invention are administered parenterally, preferably via intravenous, intramuscular or subcutaneous infusion or injection, or orally, transdermally or nasally, or by suppository, preferably vaginal suppository, or by pulmonary delivery. It is preferable that the FimH compositions not be injected intraperitoneally.

In a preferred embodiment, the FimH compositions is administered in a dose of 25 μg per adult human subject. In another embodiment, the adult human subject is given a dose of about 20 to 30 μg of the FimH compositions. In another embodiment, the adult human subject is given a dose of about 1 to 20 μg of the FimH compositions. In another embodiment, the adult human subject is given a dose of about 30 to 50 μg of the FimH compositions. In yet another embodiment, the adult human subject is given a dose of 1, 5, 50, 100 or 123 μg of the FimH compositions.

The present invention also provides methods for preventing, treating or ameliorating one or more symptoms associated with a UTI infection in a primate comprising administering to said primate a first dose of a FimH composition or an immunogenic fragment thereof, followed by administration of a second dose two weeks to one month later, and if necessary, followed by a third dose from 16 to 48 weeks following the first dose. The necessity of a third dose can be determined by one of skill in the art, preferably as being the lack of detectable secreted IgG in the urine or vaginal mucosa secretions which have specificity for FimCH.

### 3.1 Definitions

The term “analog” as used herein refers to a polypeptide that possesses a similar or identical function as a FimH polypeptide or FimCH polypeptide complex, or a fragment thereof, but does not necessarily comprise a similar or identical amino acid sequence or structure of a FimH polypeptide or FimCH polypeptide complex or a fragment thereof. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a FimH polypeptide or FimCH polypeptide complex or a fragment thereof as described herein; (b) a polypeptide encoded by a nucleotide sequence that hybridizes under stringent conditions to a nucleotide sequence encoding a FimH polypeptide or FimCH polypeptide complex or a fragment thereof as described herein of at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%o, at least 80%, at least 85%, at least 90%, at least 95% or at least 99% identical to the nucleotide sequence encoding a FimH polypeptide or FimCH polypeptide complex or a fragment thereof as described herein. A polypeptide with similar structure to a FimH polypeptide or FimCH polypeptide complex or a fragment thereof as described herein refers to a polypeptide that has a similar secondary, tertiary or quaternary structure of a FimH polypeptide or FimCH polypeptide complex or a fragment thereof as described herein. The structure of a polypeptide can determined by methods known to those skilled in the art, including but not limited to, X-ray crystallography, nuclear magnetic resonance, and crystallographic electron microscopy.

The term “derivative” as used herein refers to a polypeptide that comprises an amino acid sequence of a FimH polypeptide or FimCH polypeptide complex or a fragment thereof as described herein that has been altered by the introduction of amino acid residue substitutions, deletions or additions. The term “derivative” as used herein also refers to a FimH polypeptide or FimCH polypeptide complex or a fragment thereof that has been modified, i.e, by the covalent attachment of any type of molecule to the polypeptide. For example, but not by way of limitation, a FimH polypeptide or FimCH polypeptide complex or a fragment thereof may be modified, e.g., by glycosylation, acetylation, pegylation, phosphorylation, amidation, derivatization by known protecting/blocking groups, proteolytic cleavage, linkage to a cellular ligand or other protein, etc. A derivative of a FimH polypeptide or FimCH polypeptide complex or a fragment thereof may be modified by chemical modifications using techniques known to those of skill in the art, including, but not limited to specific chemical cleavage, acetylation, formylation, metabolic synthesis of tunicamycin, etc. Further, a derivative of a FimH polypeptide or FimCH polypeptide complex or a fragment thereof may contain one or more non-classical amino acids. A polypeptide derivative possesses a similar or identical function as a FimH polypeptide or FimCH polypeptide complex or a fragment thereof described herein.

The term “fragment” as used herein refers to a peptide or polypeptide comprising an amino acid sequence of at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a FimH polypeptide.

An “isolated” or “purified” polypeptide or polypeptide complex of the invention or fragment thereof is substantially free of cellular material or other contaminating proteins from the cell or tissue source from which the protein is derived, or substantially free of chemical precursors or other chemicals when chemically synthesized. The language “substantially free of cellular material” includes preparations of a polypeptide or polypeptide complex in which the polypeptide or polypeptide complex is separated from cellular components of the cells from which it is isolated or recombinantly produced. Thus, a polypeptide or polypeptide complex that is substantially free of cellular material includes preparations of polypeptide or polypeptide complex having less than about 30%, 20%, 10%, or 5% (by dry weight) of heterologous protein (also referred to herein as a “contaminating protein”). When the polypeptide or polypeptide complex is recombinantly produced, it is also preferably substantially free of culture medium, i.e., culture medium represents less than about 20%, 10%, or 5% of the volume of the protein preparation. When the polypeptide or polypeptide complex is produced by chemical synthesis, it is preferably substantially free of chemical precursors or other chemicals, i.e., it is separated from chemical precursors or other chemicals which are involved in the synthesis of the protein. Accordingly such preparations of the polypeptide or polypeptide complex have less than about 30%, 20%, 10%, 5% (by dry weight) of chemical precursors or compounds other than the polypeptide or polypeptide complex of interest. In a preferred embodiment, polypeptides or polypeptide complexes or fragments thereof of the invention are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

The term “periplasmic chaperone” is defined as a protein localized in the periplasm of bacteria that is capable of forming complexes with a variety of chaperone-binding proteins via recognition of a common binding epitope (or epitopes). Chaperones perform several functions. They serve as templates upon which proteins exported from the bacterial cell into the periplasm fold into their native conformations. Association of the chaperone-binding protein with the chaperone also serves to protect the binding proteins from degradation by proteases localized within the periplasm, increases their solubility in aqueous solution, and leads to their sequentially correct incorporation into an assembling pilus. Chaperone proteins are a class of proteins in gram-negative bacteria that are involved in the assembly of pili by mediating such assembly, but are not incorporated into the structure. PapD is the periplasmic chaperone protein mediating the assembly of pili for P piliated bacteria and FimC is the periplasmic chaperone protein that mediates assembly of type 1 pili in bacteria.

The term “fusion protein” as used herein refers to a polypeptide that comprises an amino acid sequence of a polypeptide or fragment thereof and an amino acid sequence of a heterologous polypeptide (e.g., FimH conjugated to FimC).

The term “attachment domain” refers to the portion of a polypeptide that mediates binding between the polypeptide and a second moiety. The second moiety can comprise cell surface polypeptides and/or polysaccharides. The attachment domain for a FimH polypeptide, which is a type 1 adhesin protein produced by E. coli, is depicted in FIG. 4. In particular, the β-sheets that make up the attachment binding domain are labeled 1-11 in FIG. 4.

The term “FimH antigen” refers to a FimH polypeptide or fragment thereof to which an antibody or antibody fragment immunospecifically binds. A FimH antigen also refers to an analog or derivative of a FimH polypeptide or fragment thereof to which an antibody or antibody fragment immunospecifically binds.

The term “FimCH complex” refers to a complex containing both a FimH and a FimC polypeptide preferably in a 1:1 ratio in the complex.

The term “antibodies or fragments that immunospecifically bind to a FimH antigen” as used herein refers to antibodies or fragments thereof that specifically bind to a FimH polypeptide or a fragment of a FimH polypeptide and do not non-specifically bind to other polypeptides. Antibodies or fragments that immunospecifically bind to a FimH polypeptide or fragment thereof may have cross-reactivity with other antigens. Preferably, antibodies or fragments that immunospecifically bind to a FimH polypeptide or fragment thereof do not cross-react with other antigens. Antibodies or fragments that immunospecifically bind to a FimH polypeptide can be identified, for example, by immunoassays or other techniques known to those of skill in the art.

The term “patient in need thereof” refers to a human that is infected with, or at risk of being infected with, pathogenic bacteria that produce pili, especially E. coli and related bacteria. This term also includes in specific embodiments, patients previously having had a UTI. Further this term includes in certain embodiments immunocompromised patients. For research purposes, a mouse model or Cynomolgus monkey can be utilized to simulate such a patient in some circumstances.

The terms “pili”, “fimbriae,” and “fibrillae” are used herein to refer to heteropolymeric protein structures located on the extracellular surface of bacteria, most commonly gram-negative bacteria. Typically these structures are anchored in the outer membrane. Throughout this specification the terms pilus, pili, fimbriae, and fibrilla will be used interchangeably.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (e.g., http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### 4. BRIEF DESCRIPTION OF THE DRAWINGS

FIG. 1 is a chart of results from an ELISA of levels of anti-FimH specific IgGs in preimmune, days 7, 28, 35, 58, 112, 119 and 140 post-FimCH vaccination serum of vaccinated human subjects. Titers are shown as endpoint dilutions which are measured by an ELISA where FimH-T3 is the capture antigen and the detection antibody is specific to IgG. Booster doses of the vaccine were given on days 28 and day 112.

FIGS. 2A-2E. FIG. 2A is a chart showing binding inhibition, measured by multiple channel fluorescence (MCF) in log2 scale, of E. coli NU-14 to human bladder cells J82, in the presence of the indicated dilutions of serum from human subjects vaccinated with the MF59C.1 adjuvant. FIG. 2B is a chart showing binding inhibition of E. coli NU-14 to human bladder cells J82, in the presence of the indicated dilutions of serum from human subjects vaccinated with 1 μg of FimCH. FIG. 2C is a chart showing binding inhibition of E. coli NU-14 to human bladder cells J82, in the presence of the indicated dilutions of serum from human subjects vaccinated with 5 μg of FimCH. FIG. 2D is a chart showing binding inhibition of E. coli NU-14 to human bladder cells J82, in the presence of the indicated dilutions of serum from human subjects vaccinated with 25 μg of FimCH. FIG. 2E is a chart showing binding inhibition of E. coli NU-14 to human bladder cells J82, in the presence of the indicated dilutions of serum from human subjects vaccinated with 123 μg of FimCH.

FIGS. 3A-3B. FIG. 3A is a graphical representation of data from an ELISA, (FimH-T3 is the capture antigen and the detection antibody is specific for IgG), which shows levels of anti-FimH specific IgGs present in the urine of preimmune or vaccinated human subjects. FIG. 3B is an ELISA showing levels of anti-FimH specific IgGs present in the vaginal secretions of preimmune or vaccinated human subjects.

FIG. 4. FIG. 4 depicts β-sheet topology diagrams for the attachment binding domain (left) and chaperone binding domain (right) of FimH. The β-sheet structures of the attachment binding domain are labeled 1-11, and the β-sheet structures of the chaperone binding domain are labeled A′-F. The assignment of these β-sheet structures is consistent with that described by Choudhury et al. (Science 285:1061 (1999)), which is incorporated by reference herein in its entirety.

### 5. DETAILED DESCRIPTION OF THE INVENTION

The present invention relates to methods of stimulating an immune response in a primate by administering bacterial adhesin proteins, or immunogenic fragments thereof, particularly peptides comprising an attachment domain of a type 1 pilin polypeptide, preferably a FimH protein, or fragment thereof that contains the attachment domain and/or binds to mannose residues. Surprisingly, such methods for stimulating an immune response result in the production of the presence of IgGs specific for the bacterial adhesin protein (particularly, IgGs that prevent binding of the bacteria to the cells of the primate) in mucosal secretions of the primate, particularly the urine and/or genital secretions, such that the incidence of the bacterial infection is reduced. Such methods may be used to prevent, treat or ameliorate the symptoms associated with infection by the bacterium associated with the adhesin protein, particularly infections of the urogenital tract, specifically UTIs.

### 5.1 Prophylactic and Therapeutic Methods

The invention provides methods of inducing immunoglobulin molecules that specifically bind a bacterial adhesin protein, preferably, an attachment domain of a type 1 pilin polypeptide, associated with a bacterium (and, preferably, also inhibit binding of the bacterium to a cell surface molecule of a tissue the bacterium infects, e.g., in the case of FimH, inhibit binding of E. coli to mannose residues) that causes urogenital tract infections, such that the infections are ameliorated, prevented or treated. The methods comprise administering to a primate an appropriate amount of a purified peptide or peptide complex of the invention which is sufficient to achieve a level of anti-adhesin protein immunoglobulin molecules in the serum and, preferably, in the urine or genital tract secretions of the primate, sufficient to reduce the incidence of, or ameliorate urogenital tract infections. Alternatively, the invention provides methods of eliciting an immune response in a primate to a polypeptide or complex thereof of the invention to induce a prophylactic level of immunoglobulin molecules in the serum, and preferably, in the urine and genital secretions of the primate resulting in a reduction in the incidence of a bacterial infection.

The methods of the invention result in prophylactic or therapeutic levels of adhesin protein-specific immunoglobulins in the serum and, preferably, in the urine and mucosal secretions of the subject. These immunoglobulins, which preferably are IgGs, but may be any type of immunoglobulin molecules, for example, but not limited to, IgAs, that specifically bind the bacterial adhesin protein, particularly the attachment domain of a type 1 pilin polypeptide, and preferably a FimH polypeptide. Methods for assaying specific binding of immunoglobulins to an antigen are well known and routine in the art. Examples of such methods are described in Section 5.4, infra.

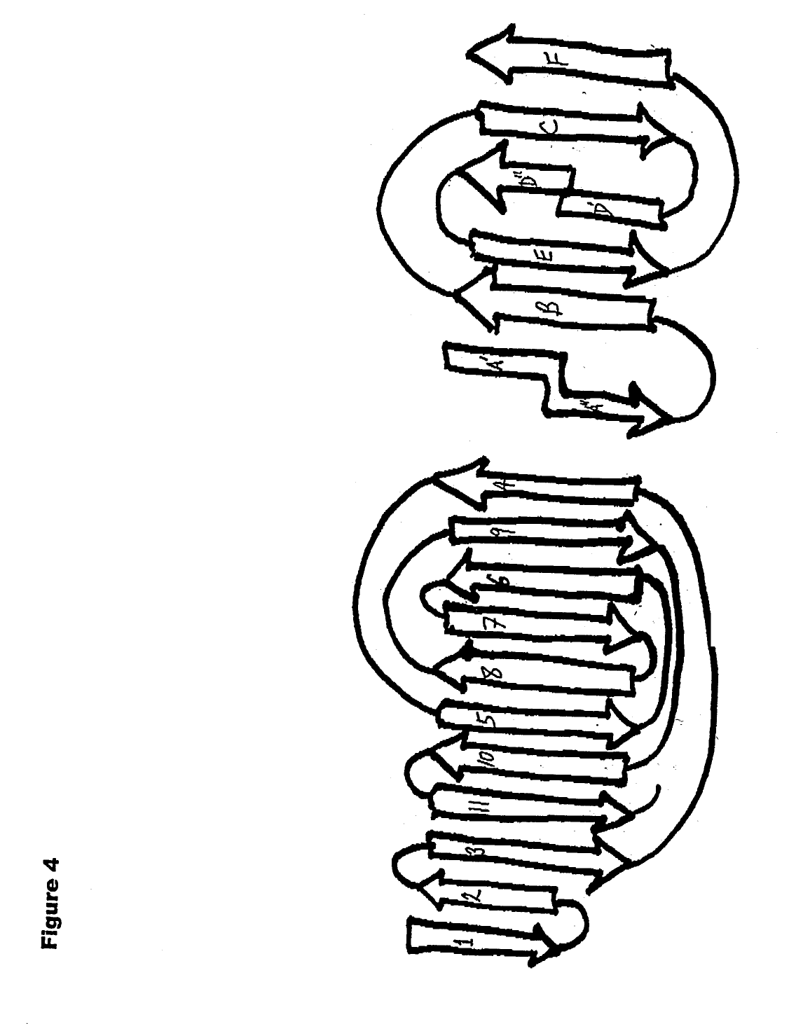

Additionally, attachment domains are the portions of the bacterial adhesin protein, preferably a type 1 pilin protein, more preferably FimH, that mediate binding of the bacteria with which it is associated to cells and, more particularly, cell surface residues, of its host. For example, the FimH type 1 pilin polypeptide of E. coli mediates binding of the E. coli to bladder epithelial cells, particularly to D-mannose residues on cell surface glycoproteins of the bladder epithelial cells. In FimH, the attachment domain is the N-terminal domain of the protein, e.g., the β-sheet structures labeled 1-11 in FIG. 4. Accordingly, the methods of the invention preferably also result in the production of immunoglobulins, particularly IgGs, in the serum and, preferably in the urine and mucosal secretions of the subject, that inhibit binding of the bacterium to host cells or cell surface moieties thereof. In the case of Fim H, the produced immunoglobulins inhibit (and/or compete for) binding of E. coli or its adhesin protein to bladder epithelial cells and/or mannose residues. In vitro methods for assaying for the ability of antibodies to inhibit E. coli binding to epithelial cells are known in the art, examples of which are described in Section 5.4, infra.

Accordingly, and in specific embodiments, the present invention provides methods of inducing an immune response, producing immunoglobulin molecules, and prophylactic and therapeutic methods, involving administration of a bacterial adhesin protein and complexes thereof which methods achieve a level of adhesin protein specific immunoglobulin molecules, preferably IgGs, in the serum of the primate and/or in the urine or mucosal secretions of the primate. These levels are sufficient to reduce the incidence of or to treat a particular bacterial infection, preferably infections of the urogenital tract. In one embodiment, the methods of the invention achieve in the serum of the primate endpoint titers of the bacterial adhesin protein specific immunoglobulins of at least 3,200, at least 12,000, more preferably, at least 20,000, at least 50,000, at least 100,000, at least 150,000, at least 200,000, or at least 300,000, however, most preferably at least 100,000. Additionally, methods of the invention achieve in the serum of the primate levels of immunoglobulin molecules that inhibit bacterial binding to cell surface proteins sufficient to achieve at least 60%, at least 75%, at least 80%, preferably at least 90%, and more preferably 100% inhibition as compared to inhibition by pre-immune serum. Methods of the invention also achieve in the serum of the subject functional inhibitory endpoint titer (i.e., the highest dilution (most dilute) that results in 50% binding inhibition as compared to pre-immune serum) of at least 1:50, at least 1:100, at least 1:200, at least 1:400, at least 1:800, preferably at least 1:1600, or at least 1:3200 using, for example, the assay described in section 5.4. In other embodiments, such levels of bacterial adhesin specific immunoglobulin molecules (either endpoint titers and/or inhibitory endpoint titers) are detected in the serum of the primate and, additionally, immunoglobulin molecules that bind the bacterial adhesin protein and/or inhibit bacterial binding are detected in the urine and/or mucosal secretions of the primate.

In a preferred embodiment, the methods of the invention induce in the urine or mucosal secretions (e.g., cervical secretions) of the primate the presence of the bacterial adhesin protein specific immunoglobulins, detected by ELISA, for example, described in Section 5.4, infra, preferably, where the levels of the immunoglobulins are at least 500, more preferably at least 1,000, at least 5,000, at least 12,000, at least 50,000, and even more preferably at least 100,000. More preferably, methods of the invention induce, in the urine or mucosal secretions of the primate, immunoglobulin molecules that inhibit bacterial binding as compared to inhibition by pre-immune serum (using, for example, the method described in Section 5.4).

The present invention encompasses the administration of a bacterial adhesin protein, preferably associated with a pathogenic bacteria. The bacterial adhesin protein is preferably a type 1 pilus polypeptide. Fragments of the bacterial adhesin protein containing, for example, all or an immunogenic portion of the attachment domain (preferably, a portion that binds cell surface residues and/or mannose) of the protein may also be administered. Such bacterial adhesin proteins also include analogs, homologs and variants thereof, preferably that retain binding activity. In other embodiments, the bacterial adhesin proteins are provided as part of a complex, for example, with a bacterial chaperone protein, as detailed below.

In preferred embodiments, the methods of the invention encompass administration of a FimH protein, including variants, derivatives, analogs and fragments thereof, preferably variants, derivatives, analogs and fragments that retain mannose binding activity and, preferably, are immunogenic. In one embodiment of the present invention, FimH proteins (naturally or recombinantly produced, as well as functional analogs) from bacteria that produce type 1 pili are contemplated. Even more particularly, E. coli FimH proteins are contemplated, preferably from E. coli strain J96, a uropathogenic isolate, having an amino acid sequence as set forth in SEQ ID No.:4, and variants, analogs, derivatives and fragments thereof.

The present invention also provides for administration of FimH polypeptides, differing only in selected amino acid locations, which polypeptides are sufficiently variable to elicit strong immune reactions but similar enough in structure to afford protection against a wide array of E. coli strains to be generally useful, such polypeptides are disclosed in co-owned U.S. application Ser. No. 09/616,702, filed Jul. 14, 2000, entitled “FimH Adhesin Based Vaccines” by Hultgren et al.; and U.S. Provisional Application No. 60/216,750, filed Jul. 7, 2000, entitled “FimH Adhesin Proteins” by Langermann et al., each of which is hereby incorporated by reference in its entirety.

Additionally, the methods and compositions of the present invention also include synthetic structures comprising non-contiguous domains of FimH and its variants. It is known that the antigenic portions of FimH are generally composed of the mannose-binding segments, formed of about the N-terminal two thirds of the molecule. The remaining pilin-binding portion is the segment that interacts with FimC to form a complex in the fibrillum of the bacterial cell. Thus, the FimH variants of the present invention are readily engineered to produce only the specific, and relatively short, mannose-binding domains of the N-terminal two thirds of the sequences. These attachment domains, known in the art, are readily strung together using convenient linker sequences, or other linking structures, to provide polypeptides composed of such non-contiguous mannose binding domains, the overall structure of which provides a highly immunogenic structure for use in the methods and compositions disclosed herein.

One problem with utilizing such proteins has been that synthesis of the polypeptide, such as FimH, results in a protein that falls short of attaining its native in vivo structure. Thus, there is a difference between the in vivo conformation of such a protein and that attained by a purified recombinant form of such protein.

The reason for this difference in conformation has been determined. In general, a pilin protein, such as an adhesin like FimH, has a native conformation that is at least partly determined by the in vivo interaction of such protein with an additional protein, here a periplasmic chaperone protein called FimC. The resulting FimC-FimH (or FimCH) complex is the form that presents the native FimH conformation as seen in vivo and thus by the immune system (Choudhury et al., X-ray Structure of the FimC-FimH Chaperone-Adhesin Complex from Uropathogenic E. coli, Science 285, 1061 (1999); Sauer et al., Structural Basis of Chaperone Function and Pilus Biogenesis, Science 285, 1058 (1999)). Consequently, the methods and compositions of the invention include such complexes where said proteins are co-expressed, or otherwise formed in a combined state, with their respective periplasmic chaperone thereby yielding the native complex normally seen in vivo by the immune system following infection by a disease causing pathogen. Accordingly, the present invention further encompasses administration of such pilin complexes, i.e., complexes of FimC with a FimH polypeptide.

FimH complexes can be readily produced by recombinant methods in such a way as to incorporate therein the sequences provided by FimC in the FimCH complex, thus yielding a native structure for FimH, which structure is immunogenic in nature. In essence, the portion of the FimC molecule that binds to FimH and directs its native conformation is engineered into the FimH structure itself, at the appropriate location, to result in a native FimH structure. This portion of the FimC molecule that binds to FimH in the FimCH complex is called a “donor strand” and the mechanism of formation of the native FimH structure using only this additional strand from FimC has been referred to as “donor strand complementation.” Thus, the FimH complexes, can be produced in their “donor complemented” form to provide highly immunogenic structures for use in therapeutically effective vaccine compositions within the present invention. Such donor strand complemented forms are disclosed in detail in U.S. application Ser. No. 09/615,846, filed Jul. 13, 2000 and PCT/US00/19066, filed Jul. 13, 2000, both entitled “Donor Strand Complemented Pilus-Based Vaccines”, each of which is hereby incorporated by reference herein in its entirety.

Accordingly, in preferred embodiments, complexes of FimH and FimC are administered in the methods of the invention. Such complexes include FimH-FimC fusion proteins and complexes, preferably, containing an equimolar ratio of FimH and FimC. Any known FimC protein can be used in such complexes. Preferably the FimC protein is from the E. coli J96 isolate and has an amino acid sequence of SEQ ID No.:2. In a more preferred embodiment, a FimCH complex containing a FimH protein and a FimC protein in equimolar amounts is administered, preferably where the FimH protein has an amino acid sequence of SEQ ID No.:4 and the FimC protein has an amino acid sequence of SEQ ID No.:2. As described infra, the FimCH complexes can be expressed from the same plasmid, preferably under the control of separate promoters, and isolated from the host cell, e.g., an E. coli host cell.

In preferred embodiments, the bacterial infection, particularly a urogenital tract infection, more particularly a UTI, to be treated or prevented, is caused by a gram negative bacterium of the family Enterobacteriaceae, especially E. coli. In other embodiments, the infection is caused by Staphylococcus saprophyticus or Staphylococcus aureus, Klebsiella spp, Proteus spp, Serratia spp, or Pseudomonas spp. In an alternative embodiment, the infection is caused by infection with unusual organisms such as parasites, e.g., Echinococcus, Schistosoma haematobium or mansoni, protozoa, e.g., Trichomonas, yeast such as Candida spp, Blastomyces spp, or Coccidioides immitis, or acid-fast organisms such as Mycobacterium tuberculosis. In preferred embodiments, the infection to be treated or prevented using the methods of the invention is a UTI, a bladder infection, or a kidney infection.

In one embodiment, the primate is a human. In another embodiment, the human subject is susceptible to a recurrence of UTI due to having had a prior UTI, particularly having had two, three or even more UTIs in one year, or has a familial susceptibility, e.g., genetic predisposition. In other embodiments, the human subject is pregnant and/or hospitalized, or is immunocomprised due, for example, to a secondary disease, such as HIV or cancer, or having undergone therapies therefor, has an HIV infection or has a cancer, or is in remission therefrom. In a specific embodiment, the human subject has asymptomatic bactourea and, in particular embodiments, also is diabetic and/or is a pregnant woman. Reduced levels of IL-6 and/or IL-8 as compared to the normal levels of IL-6 and IL-8 in pregnant women have been correlated with difficulty in clearing urinary tract infections. Thus, the invention further includes treatment of pregnant women with reduced levels of IL-6 and/or IL-8. In another specific embodiment, the subject is at risk of developing end stage renal disease; accordingly, the invention further provides a method for preventing progression to end stage renal disease.

In a preferred embodiment, the FimH compositions of the invention are administered parenterally, preferably via intramuscular, intravenous or subcutaneous injection or orally, transdermally or nasally, or, via suppository, preferably a vaginal suppository, or via pulmonary delivery. Preferably, the FimH compositions are not injected intraperitoneally.

The polypeptides of the present invention may also be present in the form of a composition. Such compositions, where used for pharmaceutical purposes, will commonly have the polypeptide of the present invention suspended in a pharmacologically acceptable diluent or excipient, or they may be in lyophilized form, for example, as in detailed in Section 5.3, infra. The polypeptides of the invention are administered in an amount effective to elicit sufficient levels of antibodies, particularly IgGs, in serum and, preferably, in mucosal secretions, such as urine and/or genital secretions, to prevent bacterial infection, e.g., to reduce the incidence of such bacterial infections, or to treat or ameliorate the symptoms of bacterial infection.

### 5.2 Protein Expression and Purification

The adhesin proteins, fragments containing the attachment domains thereof, and complexes thereof maybe produced by any method available in the art. Those skilled in the art will readily be able to purify such proteins, fragments or complexes by routine techniques.

Complexes comprising the E. coli chaperone FimC and a FimH variant of the invention may be formed by co-expressing a FimH variant polypeptide, whose amino acid and nucleotide sequences are known in the art (such as the FimH having the amino acid sequence of SEQ ID No.:4) along with a FimC variant polypeptide, whose amino acid and nucleotide sequences are known in the art (such as the FimC having the amino acid sequence of SEQ ID No.:2), from a recombinant cell.

In addition, the FimC-FimH complexes useful in vaccines can be recovered from the periplasmic spaces of cells of the indicated strains disclosed herein. These complexes are found in relatively large amounts in recombinant E. coli strains which express the FimC protein at levels in excess of those produced in wild type strains. A suitable recombinant strain is C600/pHJ9205, in which expression of FimC has been put under control of the arabinose promoter. Those skilled in the art will recognize that other promoter sequences that can be regulated easily may also be used. Of course, such cells are readily engineered to express one or more of the FimH variant polypeptides of the invention. An extract of periplasm is obtained by exposing the bacteria to lysozyme in the presence of a hypertonic sucrose solution. FimCH complexes can also be purified using conventional protein purification methods well known in the art.

In a similar manner, FimH fragments can be recombinantly produced either by having E. coli produce the full-length FimH and then fragmenting the protein or may be isolated by mannose-binding affinity purification. Thus, only fragments of the FimH protein that retain mannose binding are isolated. Preferably, such mannose-binding fragments have a label such as a his-tag included and may be purified by methods such as Nickel chromatography.

In accordance with the foregoing, FimC of E. coli is available through the American Type Culture Collection (ATCC®) as accession number Z37500. A FimH protein of E. coli is available as ATCC® Accession No. 1361011.

The polynucleotides encoding the variant protein above may have the coding sequence fused in frame to a marker sequence which allows for purification of the polypeptides of the present invention. The marker sequence may be, for example, a hexa-histidine tag supplied by a pQE-9 vector to provide for purification of the mature polypeptides fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

The proteins, chaperone/adhesin complexes and mannose-binding fragments of such proteins may be recombinantly produced in an E. coli species host. FimH may likewise be produced recombinantly by producing the appropriate donor strand complemented version of FimH, wherein the amino acid sequence of FimC that interacts with FimH in the FimCH complex is itself engineered at the C-terminal end of FimH to provide the native conformation without the need for the remainder of the FimC molecule to be present. Additionally, FimH variants may also be utilized in the form of a complex comprising isolated domains thereof, especially mannose-binding domains and fragments, which domains or fragments may be linked together, either covalently or non-covalently, utilizing linking segments, such linking segments being formed of amino acid sequences or other oligomeric structures, including simple polymer structures, to provide an overall structure exhibiting immunogenic activity.

In producing said proteins recombinantly, a preferred host is a species of bacteria that can be cultured under conditions such that the usher gene (if present) is not expressed. Further preferred is a host species that is missing the usher gene or has a defective usher gene. Even further preferred is a host which is missing the pilus proteins other than the FimH protein (and may also produce the chaperone, such as FimC). When an adhesin protein or a mannose binding fragment of such adhesin protein is to be produced in the absence of its chaperone protein (or to be separated from the chaperone after production), the adhesin protein (or fragment) may be permitted to become properly folded in the presence of its chaperone protein and is then separated from the chaperone protein.

The present invention also relates to vectors which include polynucleotides encoding one or more of the adhesin or chaperone proteins of the present invention, host cells which are genetically engineered with vectors of the invention and the production of such adhesin proteins and/or chaperone proteins by recombinant techniques in an isolated and substantially immunogenically pure form.

Host cells are genetically engineered (transduced or transformed or transfected) with the vectors comprising a polynucleotide encoding a chaperone, adhesin protein, mannose binding fragment of an adhesin protein, or the like, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the polynucleotides which encode such polypeptides. The culture conditions, such as temperature, pH and the like, are those previously used with the host cell selected for expression, and will be apparent to the ordinarily skilled artisan.

Vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as retrovirus, vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in prokaryotic cell culture, e.g., E. coli.

Optimal expression of a FimCH complex has been achieved using a newly constructed single vector containing the FimH and FimC genes but having the advantage that each gene is under its own separate lac promoter. Thus, one lac promoter is 5′ with respect to FimC while the second lac promoter is 5′ to the FimH gene. This plasmid was successfully constructed using the common plasmid pUC19 as a background vector (Yannish-Perron, C., Vierira, J. and Messing, J., Gene, 33:103-119 (1985)). This new plasmid, when used to transform the host E. coli strain BL21 (as described in Phillips, T. A., Van Bogelen, R. A., and Neidhart, F. C., J. Bacteriol. 159:283-287 (1984)) and then induced using IPTG at the mid-logarithmic stage of growth, gives maximal expression of the FimCH complex in the bacterial periplasmic space. This material is then extracted and purified by methods well known in the art, including those described herein.

The vector containing the appropriate DNA sequence as hereinabove described, as well as an appropriate promoter or control sequence, may be employed to transform an appropriate host to permit the host to express the proteins.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

Constructs for production of the adhesin proteins comprise a vector, such as a plasmid or viral vector, into which a sequence of the invention has been inserted, in a forward or reverse orientation. The construct may further comprise regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9 (Qiagen, Inc.), pbs, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia). Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host. Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and TRP. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The host cell for recombinant production can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology, (1986)).

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts, as well as other methods in molecular biology, are described in Sambrook, et al., Molecular Cloning: A Laboratory Manual, Second Edition, Cold Spring Harbor, N.Y., (1989), Wu et al., Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP 1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences. Optionally, the heterologous sequence can encode a fusion protein including an N-terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, a french press, mechanical disruption, or use of cell lysing agents, such methods are well know to those skilled in the art.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The polypeptides can be recovered and/or purified from recombinant cell cultures by well-known protein recovery and purification methods. Such methodology may include ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. In this respect, chaperones may be used in such a refolding procedure. Finally, high performance liquid chromatography (HPLC) can be employed for final purification steps.

The polypeptides that are useful as immunogens in the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Particularly preferred immunogens are FimH adhesin protein or mannose-binding fragments thereof since FimH is highly conserved among many bacterial species. Therefore, antibodies against FimH (or its mannose-binding fragments) should bind to FimH of other bacterial species (in addition to E. coli) and vaccines against E. coli FimH (or FimH mannose-binding fragments) should give protection against other bacterial infections in addition to E. coli infections (for example, against other Enterobacteriacea infections) (see, e.g., U.S. application Ser. No. 09/615,846 and PCT application No. PCT/US00/19066, both entitled “Donor Strand Complemented Pilus-Based Vaccines” and filed Jul. 13, 2000; U.S. application Ser. No. 09/616,702, filed Jul. 14, 2000, entitled “FimH Adhesin Based Vaccines” by Hultgren et al.; and U.S. Provisional Application No. 60/216,750, filed Jul. 7, 2000, entitled “FimH Adhesin Proteins” by Langermann et al.)

Procedures for the isolation of a periplasmic chaperone protein complexed with an adhesin protein are known in the art, as an example see Jones et al., (Proc. Natl. Acad. Sci. 90:8397-8401 (1993)). Further, the individually expressed adhesin proteins may be isolated by recombinant expression/isolation methods that are well-known in the art. Typical examples for such isolation may utilize an antibody to the protein or to a His tag or cleavable leader or tail that is expressing as part of the protein structure.

The FimCH polypeptides useful in forming the vaccine compositions of the present invention may conveniently be cloned using various cloning systems. An example of a useful cloning system for synthesizing FimCH is presented in Section 6 and utilizes a plasmid based cloning system. The FimCH complex described therein is composed of a 52 kDa complex composed of two proteins: FimC (22.8 kDa) and FimH (29.1 kDa) in a 1:1 equimolar ratio. The FimCH complex is expressed from a pUC-based vector (pGCA139-1-1) with two separate lac-inducible promoters driving expression of the FimC and FimH genes, respectively. The FimC and the FimH genes in the pGCA139-1-1 vector were derived from uropathogenic E. coli isolate J96 and have the nucleotide sequences of SEQ ID Nos.:1 and 3, respectively.

The FimCH complex is produced in the periplasm of E. coli strain BL21 and is purified from periplasmic extracts by standard chromatographic methods. The FimCH protein has been formulated in a number of different buffers compatible with its solubility profile including 20 mM HEPES (pH 7.0), PBS (pH 7.0) and sodium citrate (pH 6.0) in 0.2 M NaCl. This sodium citrate/sodium chloride formulation enhances the stability of the FimCH complex and is also compatible with commonly used diluents.

Plasmid pCGA139-1-1 was constructed as a means of producing relatively large amounts of E. coli chaperone-adhesin complex, FimCH, for use in the vaccine compositions disclosed herein.

The plasmid vector, pCGA139-1-1, contains the following genetic elements: (1) an E. coli FimC chaperone gene followed by (2) the FimH adhesin gene, both from E. coli strain J96 (a urinary tract infection (UTI) isolate) each preceded by its respective native signal sequence (nss); (3) a kanamycin resistance (kan^{r} or k^{r}) marker; (4) lac^{q} which codes for a repressor protein that binds the lac promoter unless it is induced; (5) an inactivated beta-lactamase (bla) gene; (6) pUC origin of replication (or); and (7) two lac promoters, one preceding the FimC signal and the other preceding that of FimH.

### 5.3 Pharmaceutical Formulations and Administration

The bacterial adhesin polypeptides and fragments thereof described herein are useful immunogens for preparing pharmaceutical compositions that stimulate the production of antibodies that confer immunity to pathogenic species of bacteria, in particular bacteria that are responsible for causing urinary tract infections.

The pharmaceutical compositions useful herein also contain a pharmaceutically acceptable carrier, including any suitable diluent or excipient, which includes any pharmaceutical agent that does not itself induce the production of antibodies harmful to the primate receiving the composition, and which may be administered without undue toxicity.

In preferred embodiments, the pharmaceutical formulations of the invention comprise a FimH polypeptide, FimCH polypeptide complex or fragments or variants thereof, and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in REMINGTON'S PHARMACEUTICAL SCIENCES (Mack Pub. Co., N.J. current edition). The formulation should suit the mode of administration. In a preferred embodiment, the formulation is suitable for administration to humans, preferably is sterile, non-particulate and/or non-pyrogenic. In a preferred embodiment the pharmaceutical composition contains a citrate buffer, preferably, about 20 mM sodium citrate and 0.2 M NaCl, more preferably with a pH of 6.0.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

The invention provides in one embodiment a thermally stable and/or chemically stable pharmaceutical composition that is suitable for reconstitution into an injectable sterile and particulate-free solution.

The invention also provides a pharmaceutical pack or kit comprising one or more containers filled with one or more of the ingredients of the vaccine formulations of the invention. In a preferred embodiment, the kit comprises two containers, one containing the adhesin protein or protein complex and the other containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that a FimH polypeptide, FimCH polypeptide complex or fragments thereof are packaged in a hermetically sealed container such as an ampoule or sachette indicating the quantity of composition. In one embodiment, the FimH composition is supplied as a liquid, in another embodiment, as a dry sterilized lyophilized powder or water free concentrate in a hermetically sealed container and can be reconstituted, e.g., with water or saline to the appropriate concentration for administration to a subject. Preferably, the FimH composition is supplied as a dry sterile lyophilized powder in a hermetically sealed container at a unit dosage of preferably, 1 μg, 5 μg, 10 μg, 20 μg, 25 μg, 30 μg, 50 μg, 100 μg, 123 μg, 150 μg, or 200 μg. Alternatively, the unit dosage of the FimH composition is less than 1 μg, (for example 0.5 μg or less, 0.25 μg or less, or 0.1 μg or less), or more than 123 μg, (for example 150 μg or more, 250 μg or more, or 500 μg or more).

The FimH composition should be administered within 12 hours, preferably within 6 hours, within 5 hours, within 3 hours, or within 1 hour after being reconstituted from the lyophylized powder.

In an alternative embodiment, a FimH polypeptide or fragment thereof is supplied in liquid form in a hermetically sealed container indicating the quantity and concentration of the FimH compositions. Preferably, the liquid form of the FimH polypeptide or fragment thereof is supplied in a hermetically sealed container at least 50 μg/ml, more preferably at least 100 μg/ml, at least 200 μg/ml, at least 500 μg/ml, at least 1 mg/ml, and most preferably 490 μg/ml.

In a preferred embodiment, FimCH is stored in a 3 mL sterile vial containing 1.0 mL of vaccine formulated in 500 μg/mL of FimCH in 20 mM sodium citrate, 0.2 M NaCl at a pH of 6.0. In this formulation, the vial should contain a clear colorless liquid. The adjuvant is stored in a separate 3 mL vial containing 0.7 mL of adjuvant (MF59C. 1; 39 mg/mL squalene, 4.7 mg/mL each Tween 80 and Span 85, 10 mM citrate in sterile water for injection at pH 6.5) and is typically a cloudy, white, turbid liquid. The diluent is supplied in another separate 3 mL vial containing 2.0 mL of 20 mM sodium citrate, 0.2 M NaCl at a pH of 6.0. The diluent is a clear, colorless liquid. Each of these vials should be stored in a refrigerator (2° C. to 8° C./36° F. to 46° C.). In a preferred embodiment, FimCH is prepared for injection into a subject immediately prior to the injection, i.e., mixed with diluent and adjuvant.

Doses of 1 μg, 5 μg, 25 μg and 123 μg of FimCH are preferably prepared for administration as follows:

For a 1 μg dose, gently invert several times one FimCH vaccine vial, three diluent vials and one adjuvant vial and let stand at room temperature for twenty minutes. Withdraw 0.5 ml from the FimCH vial into a 1.0 ml syringe and inject into a diluent vial. Immediately mix by gently swirling. Withdraw 0.5 ml using a new needle and inject into a second diluent vial. Immediately mix by gently swirling. Withdraw 0.5 ml using a new needle and inject into the third diluent vial. Immediately mix by gently swirling. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 1 μg of FimCH and MF59C.1 (approximately 10 mg squalene) in 15 mM sodium citrate and 0.1 M NaCl.

For a 5 μg dose, gently invert several times one FimCH vaccine vial, three diluent vials and one adjuvant vial and let stand at room temperature for twenty minutes. Withdraw 0.5 ml using a new needle and inject into a second diluent vial. Immediately mix by gently swirling. Withdraw 0.5 ml using a new needle and inject into the third diluent vial. Immediately mix by gently swirling. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the, needle), label syringe and place in the labeled zip-lock bag. This 0.5 ml dose will contain approximately 5 μg of FimCH and MF59C.1 (approximately 10 mg squalene) in 15 mM sodium citrate and 0.1 M NaCl.

For a 25 μg dose, gently invert several times one FimCH vaccine vial, three diluent vials and one adjuvant vial and let stand at room temperature for twenty minutes. Withdraw 0.5 ml using a new needle and inject into the third diluent vial. Immediately mix by gently swirling. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 25 μg of FimCH and MF59C.1 (approximately 10 mg squalene) in 15 mM sodium citrate and 0.1 M NaCl.

For a 123 μg dose, gently invert several times one FimCH vaccine vial, three diluent vials and one adjuvant vial and let stand at room temperature for twenty minutes. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 123 μg of FimCH and MF59C.1 (approximately 10 mg squalene) in 15 mM sodium citrate and 0.1 M NaCl.

In another specific embodiment, 1, 5, 25 or 123 μg of FimCH in 0.5 mL of MF59C.1, as prepared above, is injected slowly, i.e., 20 to 30 seconds, into the deltoid muscle of the upper arm of the subject at day 0, followed by a booster dose approximately one month, and a second booster, if necessary approximately six months, after the initial administration. The necessity of booster shots can be determined by measuring serum, urine or mucosal secretions for immunoglobulins specific to FimH.

### 5.3.1 Adjuvants

The invention encompasses bacterial adhesin protein, e.g., fimH compositions, for use in vaccines administered in conjunction with adjuvants, wherein the adjuvants can be mixed (before or simultaneously upon injection) with the FimH composition or alternatively the adjuvant is not mixed with the FimH composition but is separately co-administered with the FimH composition.

FimH compositions are administered with one or more adjuvants. In one embodiment, the FimH composition is administered together with a mineral salt adjuvants or mineral salt gel adjuvant. Such mineral salt and mineral salt gel adjuvants include, but are not limited to, aluminum hydroxide (ALHYDROGEL, REHYDRAGEL), aluminum phosphate gel, aluminum hydroxyphosphate (ADJU-PHOS), and calcium phosphate.

In another embodiment, the FimH composition is administered with an immunostimulatory adjuvant. Such class of adjuvants, include, but are not limited to, cytokines (e.g., interleukin-2, interleukin-7, interleukin-12, granulocyte-macrophage colony stimulating factor (GM-CSF), interferon-γ, interleukin-1β (1L-1β), and IL-1β peptide or Sclavo Peptide), cytokine-containing liposomes, triterpenoid glycosides or saponins (e.g., QuilA and QS-21, also sold under the trademark STIMULON, ISCOPREP), Muramyl Dipeptide (MDP) derivatives, such as N-acetyl-muramyl-L-threonyl-D-isoglutamine (Threonyl-MDP, sold under the trademark TERMURTIDE), GMDP, N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, muramyl tripeptide phosphatidylethanolamine (MTP-PE), unmethylated CpG dinucleotides and oligonucleotides, such as bacterial DNA and fragments thereof, LPS, monophosphoryl Lipid A (3D-MLAsold under the trademark MPL), and polyphosphazenes.

In another embodiment, the adjuvant used is a CpG adjuvant. Oligo-deoxynucleotides (ODN) containing unmethylated CpG dinucleotides within specific sequence contexts (CpG motifs) are detected, like bacterial or viral DNA, as a danger signal by the vertebrate immune system. CpG ODN synthesized with a nuclease-resistant phosphorothioate backbone have been shown to be a potent Th1-directed adjuvant in mice. In addition, an ODN with a TpC dinucleotide at the 5′ end followed by three 6 mer CpG motifs (5′-GTCGTT-3′) separated by TpT dinucleotides has shown high immunostimulatory activity for human, chimpanzee, and rhesus monkey leukocytes (Hartmann et al., J. Immun, 164: 1617-1624 (2000)).

In another embodiment, suitable adjuvants include, but are not limited to: aluminim hydroxide, N-acetyl-muramyl-L-threonyl-D-isoglutamine (thr-MDP), -acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine.

In another embodiment, the adjuvant used is a particulate adjuvant, including, but not limited to, emulsions, e.g., squalene or squalane oil-in-water aduvant formulations, such as SAF and MF59, e.g., prepared with block-copolymers, such as L-121 (polyoxypropylene/polyoxyethylene) sold under the trademark PLURONIC L-121, Liposomes, Virosomes, cochleates, and imune stimulating complex, which is sold under the trademark ISCOM. In a preferred embodiment, the adjuvant is MF59, MF59C or most preferably MF59C.1 or a derivative thereof (Chiron, Emeryville, Calif.). Freund's Complete Adjuvant and Freund's Incomplete Adjuvant are also commonly used adjuvants in test animals, however these adjuvants are less preferred in primates, in particular for use in humans.

In another embodiment, a microparticulate adjuvant is used. Microparticulate adjuvants include, but are not limited to biodegradable and biocompatible polyesters, homo- and copolymers of lactic acid (PLA) and glycolic acid (PGA), poly(lactide-co-glycolides) (PLGA) microparticles, polymers that self-associate into particulates (poloxamer particles), soluble polymers (polyphosphazenes), and virus-like particles (VLPs) such as recombinant protein particulates, e.g., hepatiis B surface antigen (HbsAg).

Yet another class of adjuvants that may be used include mucosal adjuvants, including but not limited to heat-labile enterotoxin from Escherichia coli (LT), cholera holotoxin (CT) and cholera Toxin B Subunit (CTB) from Vibrio cholerae, mutant toxins (e.g. LTK63 and LTR72), microparticles, and polymerized liposomes. Additional examples of mucous targeting adjuvants are E. coli mutant heat-labile toxin LT's with reduced toxicity, live attenuated organisms that bind M cells of the gastrointestinal tract, such as V cholera and Salmonella typhi, Mycobacterium bovis (BCG), in addition to mucosal targeted particulate carriers such as phospholipid artificial membrane vesicles, copolymer microspheres, lipophilic immune-stimulating complexes and bacterial outer membrane protein preparations (proteosomes).

In other embodiments, any of the above classes of adjuvants may be used in combination with each other or with other adjuvants. For example, non-limiting examples of combination adjuvant preparations that can be used to administer the FimH compositions of the invention include liposomes containing immunostimulatory protein, cytokines, or T-cell and/or B-cell peptides, or microbes with or without entrapped IL-2 or microparticles containing enterotoxin. Other adjuvants known in the art are also included within the scope of the invention (Vaccine Design: The Subunit and Adjuvant Approach, Chap. 7, Michael F. Powell and Mark J. Newman (eds.), Plenum Press, New York, 1995, which is incorporated herein in its entirety).

The effectiveness of an adjuvant may be determined by measuring the induction of specific antibodies directed against the FimH composition formulated with the particular adjuvant. In a preferred embodiment, the adjuvant MF59C.1 is mixed with the vaccine composition, and MF59C.1 is at a dose of approximately 10 mg squalene, in 15 mM sodium citrate and 0.1 M NaCl.

### 5.3.2 Vaccine Administration

Vaccines are generally administered parenterally using methods known in the art, however, many methods of administration may be used including but not limited to oral, intradermal, intramuscular, intravenous, subcutaneous, transdermal, intranasal routes, via pulmonary delivery, via suppository, e.g., vaginal suppository, via scarification (scratching through the top layers of skin, e.g., using a bifurcated needle). In a preferred embodiment, the vaccine is administered intramuscularly. In yet another embodiment, administration is not intraperitoneal due to the substantial risks of first pass hepatic removal of the polypeptides and also because of risk of infection and adhesions.

Various delivery vehicles are known and can be used to administer the FimH compositions of the invention or fragments thereof, e.g., encapsulation in liposomes, microparticles, microcapsules, recombinant cells capable of expressing the FimH compositions, receptor-mediated endocytosis (see, e.g., Wu and Wu, J. Biol. Chem. 262:4429-4432 (1987)), construction of a nucleic acid as part of a retroviral or other vector, for example, the pCGA139-1-1 vector as described herein which can be administered as a DNA vaccine or alternatively, the nucleic acid vector can be introduced into a host cell such that the host cell expresses and secretes the vaccine composition, e.g., the FimCH polypeptide complex, and the host cell is subsequently implanted into the subject contained within a membrane suitable for human implantation.

Methods of administering a polypeptide or fragment thereof, or pharmaceutical composition include, but are not limited to, parenteral administration (e.g., intradermal, intramuscular, intravenous and subcutaneous), epidural, and mucosal (e.g., intranasal and oral or pulmonary routes or by vaginal suppositories). In a specific embodiment, compositions of the present invention or fragments thereof are administered intramuscularly, intravenously, subcutaneously, or transdermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. Administration can be systemic or local.

In yet another embodiment, the vaccine composition is administered in such a manner as to target mucous tissues in order to elicit an immune response at the site of immunization. For example, mucosa tissues such as gut associated lymphoid tissue (GALT) can be targeted for immunization by using oral administration of compositions which contain adjuvants with particular mucosa targeting properties. Additional mucosal tissues can also be targeted, such as nasopharyngeal lymphoid tissue (NALT) and bronchial-associated lymphoid tissue (BALT) (Langermann, Seminars in Gast. Dis., 7:12-18 (1996); Wizemann et al., Emerging Inf. Dis., 5:395-403 (1999); Service, Science, 265:1522-1524 (1994)).

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a an antibody of the invention or fragment thereof, care must be taken to use materials to which the FimH compositions does not absorb.

In another embodiment, the composition can be delivered in a vesicle, in particular a liposome (Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp.317-327; see generally ibid.).

In yet another embodiment, the composition can be delivered in a controlled release system. In one embodiment, a pump maybe used (Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used (e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. In yet another embodiment, a controlled release system can be placed in proximity of the therapeutic target, e.g., the urogenital tract, thus requiring only a fraction of the systemic dose (e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the composition of the invention is a nucleic acid encoding a FimH, a FimCH or a fragments thereof, the nucleic acid can be administered in vivo to promote expression of its encoded FimH compositions, by constructing it as part of an appropriate nucleic acid expression vector and administering it so that it becomes intracellular, e.g., by use of a retroviral vector (U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (e.g., Joliot et al., 1991, Proc. Natl. Acad. Sci. USA 88:1864-1868), etc. Alternatively, a nucleic acid can be introduced intra-cellularly and incorporated within host cell DNA for expression by homologous recombination.

Accordingly, also provided by the invention is a method for vaccinating a primate against urogenital tract infection, which method comprises administering to the primate a purified nucleic acid containing a nucleotide sequence encoding a peptide or peptide complex comprising a bacterial type 1 pilin attachment domain of a type 1 pilin polypeptide associated with a bacterium that causes a urogenital tract infection, said nucleic acid being administered in an amount effective to produce immunoglobulin molecules that specifically bind the type 1 pilin attachment domain. Pharmaceutical compositions containing nucleic acids comprising nucleotide sequences encoding bacterial adhesin proteins, or fragments or complexes thereof, are also provided.

The dosage of the pharmaceutical formulation can be determined readily by the skilled artisan, for example, by first identifying doses effective to elicit a prophylactic or therapeutic immune response, e.g., by measuring the serum titer of vaccine specific immunoglobulins or by measuring the inhibitory ratio of serum samples, or urine samples, or mucosal secretions. In particular, doses that result in serum endpoint titers of at least 1:800, at least 1:1600, or at least 1:3200 and/or, which have at least 50% binding inhibition of E. coli to bladder cells, upon sample dilutions of at least 1:50, at least 1:100, at least 1:200, at least 1:400, at least 1:800, at least 1:1600, or at least 1:3200, and most preferably at least 1:1600, or have detectable specific and, preferably inhibitory immunoglobulins in urine or mucosal secretions, as taught in Sections 5.4 and 6, infra, in an animal model, such as a Cynomolgus monkey, before identifying the optimal dosage in humans.

In preferred embodiments, a dose of the purified FimCH complex of 1 μg, 5 μg, 10 μg, 20 μg, 30 μg, 50 μg, 75 μg, 100 μg, 123 μg, 150 μg, or 200 μg, or preferably 25 μg is administered. In other embodiments, the dosage is in the range of 0.25 μg to 1 μg, 1 μg to 5 μg, 1 μg to 10 μg, 1 μg to 20 μg, 1 μg to 50 μg, 1 μg to 75 μg, 1 μg to 100 μg, 1 μg to 150 μg, 1 μg to 200 μg, 5 μg to 10 μg, 10 μg to 15 μg, 10 μg to 20 μg, 15 μg to 25 μg, 20 μg to 30 μg, 30 μg to 50 μg, 25 μg to 75 μg, 50 μg to 100 μg, 75 μg to 125 μg, 50 μg to 125 μg, 50 μg to 200 μg, or 100 μg to 200 μg. For pediatric uses, a fractional dose of the pharmaceutical composition may be administered. For adult patients or patients with persistent infections, larger doses may also be used.

Vaccines of the invention may also be administered on a dosage schedule, for example, an initial administration of the vaccine composition with subsequent booster administrations. In particular embodiments, a second dose of the pharmaceutical composition is administered anywhere from two weeks to one year, preferably from one to six months, after the initial administration. Additionally, a third dose may be administered after the second dose and from three months to two years, or even longer, preferably 4 to 6 months, or 6 months to one year after the initial administration. The third dose may be optionally administered when no or low levels of specific immunoglobulins are detected in the serum and/or urine or mucosal secretions of the subject after the second dose. In a preferred embodiment, a second dose is administered approximately one month after the first administration and a third dose is administered approximately six months after the first administration. In another preferred embodiment, the second dose is administered six months after the first administration.

### 5.4 Determination of Vaccine Efficacy

Immunopotency of the pharmaceutical formulations can be determined by monitoring the immune response of a subject following immunization with a bacterial adhesin composition, in particular the generation of immunoglobulins, particularly IgGs, which are detectable in the urine or mucosal secretions of the subject. Generation of a humoral response may be taken as an indication of a generalized immune response, other components of which, particularly cell-mediated immunity, may be important for protection against UTI.

Subjects can include any primate including Cynomolgus monkeys, chimpanzees and human subjects in well controlled clinical settings. In addition, bacteria causing UTI can be used to induce infection in primates experimentally. However, since many primates are a protected species, the antibody response to a vaccine of the invention can first be studied in a number of smaller, less expensive animals, with the goal of finding one or two best candidate viruses or best combinations of viruses to use in primate efficacy studies. As one example, UTI vaccines of the invention may be tested first in mice for the ability to induce an antibody response to bacterial adhesin polypeptides or polypeptide complexes and to protect against bacterial challenge.

The methods of introduction of the vaccine in the test subjects may include oral, intradermal, intramuscular, intravenous, subcutaneous, intranasal or any other standard routes of immunization.

The immune response of the test subjects can be analyzed by various approaches such as: the reactivity of the resultant immune serum or urine or mucosal secretions to E. coli pilus, as assayed by known techniques, e.g., enzyme linked immunosorbent assay (ELISA), immunoblots, radio-immunoprecipitations, etc.; or protection from UTI infections and/or attenuation of UTI symptoms in immunized hosts, for example, but not limited to; cystitis; or inhibition of binding of E. coli to cell surface residues, particularly mannose residues.

Urine and mucosa samples may be taken from the test subject every one or two weeks, and serum analyzed for antibodies to E. coli Type 1 pilus using, e.g., a radioimmunoassay (Abbott Laboratories). The presence of antibodies specific for FimH may be assayed using an ELISA. The test subject's sera may also be analyzed for antibodies to E. coli, e.g., in an enzyme-linked immunoassay.

Cynomolgus monkeys (Macaca fascicularis) may be used to test for immunogenicity of FimH vaccine formulations of the invention. In a specific embodiment, monkeys each receive intramuscularly approximately 100 μg or other appropriate dose of the adhesin in adjuvant. A control Cynomolgus monkey receives adjuvant alone. Blood is drawn weekly for 12 weeks, and serum is analyzed for antibodies to the adhesin and urine and vaginal samples are taken to assess, by ELISA or other antibody detection tests, particularly IgG secretion.

Furthermore, the antibodies that are produced in response to the vaccine can be assessed for functional activity, e.g., binding to the adhesin or inhibiting binding of type I pilin bacteria to urogenital tract cells.

A non-limiting example of a binding inhibition assay is as follows. Type 1 piliated NU14 E. coli are directly labeled with fluorescein isothiocyanate FITC) and incubated with J82 bladder cells at a ratio of 250 bacteria/cell in the presence of preimmune or immunized serum and incubated for 30 minutes at 37° C. After multiple washes, samples are assayed by flow cytometry, and percent inhibition of bacterial binding to the cells is determined. The samples, such as serum samples, urine samples or vaginal wash samples, are diluted at 1:2, 1:4, 1:8, up to 1:3200 or more, and compared relative to preimmune samples from each subject, in order to identify an endpoint dilution where the binding inhibition is equal to or less than 50%. The binding ratio is defined as the ratio of the number of bacteria or the mean channel fluorescent (MCF) value which correlates with the number of bacteria (e.g. NU 14) bound to a cell (e.g., J82) in the presence of a diluted sample from an immunized subject, relative to the number of bacteria which bind a cell in the presence of preimmune sample from a non-immunized subject.

Another non-limiting example of a binding inhibition assay is as follows. Briefly, Immulon-4 plates (Dynex Technologies, Inc., Chantilly, Va.) are coated with 2.5 μg/ml (100 ml/well) of tri-mannose-BSA (V-Labs, Covington, La.). Type 1-piliated NU14 E. coli are added to each well, incubated at 37° C. for 1 hour and after extensive washing, bound bacteria are detected with a 1:400 dilution of an anti-E. coli-HRP conjugated antibody (Biodesign, Kennebunk, Me.). OD₄₀₅ readings of these samples establish the full signal values (FSV) for binding to trimannose (approximately 2.0). Additional samples are run in the presence of 1:50 dilutions of serum to assess inhibition, where percent inhibition equals the FSV−the sample value/FSV×100. All samples are run in triplicate.

### 5.5 Anti-FimH Antibodies Generated by the Vaccines of the Invention

Antibodies generated against FimH by immunization with the vaccines formulations of the present invention also have potential uses in diagnostic immunoassays, passive immunotherapy, and generation of antiidiotypic antibodies.

The antibodies generated by the vaccine formulations of the present invention can also be used in the production of antiidiotypic antibody. The antiidiotypic antibody can then in turn be used for immunization, in order to produce a subpopulation of antibodies that bind the initial antigen of the pathogenic microorganism (Jerne, 1974, Ann. Immunol. (Paris) 125c:373; Jerne, et al., 1982, EMBO J. 1:234).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

The vaccine formulations of the present invention can also be used to produce antibodies for use in passive immunotherapy, in which short-term protection of a host is achieved by the administration of pre-formed antibody directed against a heterologous organism (in this case, FimH, FimCH or fragments thereof.

More particularly, an isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of a type 1 pilin attachment domain, and encompasses an epitope of a type 1 pilin attachment domain of the protein such that an antibody raised against the peptide forms a specific immune complex with the protein.

Preferred epitopes encompassed by an antigenic peptide are regions that are located on the surface of the protein, e.g., hydrophilic regions. In certain embodiments, the nucleic acid molecules of the invention are present as part of nucleic acid molecules comprising nucleic acid sequences that contain or encode heterologous (e.g., vector, expression vector, or fusion protein) sequences. These nucleotides can then be used to express proteins which can be used as immunogens to generate an immune response, or more particularly, to generate polyclonal or monoclonal antibodies specific to the expressed protein.

An immunogen typically is used to prepare antibodies by immunizing a suitable subject, (e.g., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g. a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

An antibody directed against a polypeptide of the invention can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In addition, type 1 pilin attachment domain gene sequences and gene products, including peptide fragments, as well as specific antibodies thereto, can be used for construction of fusion proteins to facilitate recovery, detection, or localization of another protein of interest.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells, and in particular, prokaryotic cells.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), interleukin-10 (“IL-10”), interleukin-12 (“IL-12”), interferon-γ (“IFN-γ”), interferon-α (“IFN-α”), or other immune factors or growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

An antibody with or without a therapeutic moiety conjugated to it can be used as a therapeutic that is passively administered alone or in combination with chemotherapeutic agents.

Alternatively, an antibody of the invention can be conjugated to a second antibody to form an “antibody heteroconjugate” as described by Segal in U.S. Pat. No. 4,676,980 or alternatively, the antibodies can be conjugated to form an “antibody heteropolymer” as described in Taylor et al., in U.S. Pat. Nos. 5,470,570 and 5,487,890.

An antibody with or without a therapeutic moiety conjugated to it can be used as a therapeutic that is administered alone or in combination with cytotoxic factor(s) and/or cytokine(s).

In yet a further aspect, the invention provides substantially purified antibodies or fragments thereof, including human or non-human antibodies or fragments thereof, which antibodies or fragments specifically bind to a attachment domain of a type 1 pilin polypeptide of the invention. In various embodiments, the substantially purified antibodies of the invention, or fragments thereof, can be human, non-human, chimeric and/or humanized antibodies.

In another aspect, the invention provides non-human antibodies or fragments thereof. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof. The monoclonal antibodies can be human, humanized, chimeric and/or non-human antibodies.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

After immunization, a sample is collected from the mammal that contains an antibody that specifically recognizes the immunogen. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

### 6. EXAMPLES

### 6.1 Vector preparation and FimCH Expression

The FimCH vaccine disclosed herein is made up of an approximately 52 kDa complex composed of two proteins, FimC (22.8 kDa) and FimH (29.1 kDa) in a 1:1 equimolar ratio. The FimCH complex is expressed from a pUC-based vector (pCGA139-1-1) with two separate lac-inducible promoters driving expression of the FimC and FimH genes respectively. The FimC and FimH genes in the pCGA139-1-1 vector were derived from a well-characterized uropathogenic E. coli isolate J96.

The FimCH complex is produced in the periplasm of E. coli strain BL21 and is purified from periplasmic extracts by standard chromatographic methods. The FimCH protein has been formulated in a number of different buffers compatible with its solubility profile including 20 mM HEPES (pH 7.0), PBS (pH 7.0), and 20 mM sodium citrate at pH 6.0 in 0.2 M NaCl. The sodium citrate formulation used in the FimCH vaccine product enhances stability of the FimCH complex and is also compatible with commonly used diluents as well as adjuvants, including the MF-59, MF-59C or MF-59C.1 adjuvant (Chiron, Emeryville, Calif.).

Brief Description of the pCGA139-1-1 Vector

The plasmid vector, pCGA139-1-1, contains the following genetic elements: (1) an E. coli FimC chaperone gene followed by (2) the FimH adhesin gene, both from E. coli strain J96 [a urinary tract infection (UTI) isolate] each preceded by its respective native signal sequence (nss); (3) two lac promoters, one preceding the fimC signal and the other preceding that of fimH. (4) lac^{q} which codes for a repressor protein that binds the lac promoter unless it is induced; (5) a kanamycin resistance (kan^{r} or kr) marker; (6) an inactivated beta-lactamase (bla) gene; and (7) pUC origin of replication (ori) which allows for replication of the plasmid as an episomal DNA in E. coli and dictates the plasmid copy number (pcn).

Construction of the Plasmid Vector pCGA139-1-1

The following describes the steps used in generating and optimizing the FimCH expressing vector pCGA139-1-1.

Step 1

Generation of a Vector with a lac Inducible FimC (pCGA101-8)

Genomic DNA was prepared from E coli strain J96. The pellet from 1.0 ml of an overnight culture was washed with PBS, resuspended in 500 ml sterile sucrose Tris EDTA, and 0.01 mg lysozyme was added. The suspension was incubated at approximately 37° C. for approximately 10 minutes and SDS was added to a final concentration of approximately 0.5%. The mixture was then treated with RNase for approximately 10 minutes at approximately 37° C. after which the DNA was phenol extracted and ethanol precipitated. The resulting pellet was washed with 70% ethyl alcohol, dried and resuspended in a solution containing 10.0 mM Tris and 0.1 mM EDTA. This DNA was used as template for PCR production of the FimC.

PCR was performed on genomic DNA with fimC-specific primers GA1F and GA2R containing NcoI and BglIII/SalI restriction sites respectively.

Conditions for the PCR reaction were as follows. Samples were run for 1 cycle at approximately 95° C. for approximately 1.0 minute, followed by 25 cycles of strand separation at approximately 95° C. for approximately 30 seconds, annealing at approximately 50° C. for about 30 seconds and strand elongation at approximately 72° C. for approximately 2 minutes. This was followed by one approximately 10 minute cycle at 72° C. to ensure complete elongation of all ends. PCR products were purified on Qiagen columns and the gene was cloned into the vector pPW19R, as a NcoI/SalI fragment downstream of the lac promoter, and 3′ to the Pel B leader sequence on the plasmid. The result was plasmid pCGA101-8.

Step 2

Cloning of kan^{r}, lac^{q} and lac inducible fimH and three-way ligation to generate pCGA122-30

A kanamycin resistance gene was excised as a AlwNI/StyI fragment from the vector pET26b(+) (Novagen) and cloned into the unique DraII site in a vector called pTTQ18 (Stark, 1987) 5′of the lacI^{q} gene resulting in pTTQ18K. This plasmid contains the lac^{q} and kan^{r} genes in tandem so they can be cloned as a single cassette.

FimH was cloned with its native signal sequence preceded by the lac promoter utilizing overlapping PCR and ligation. Primary PCR segments were generated as follows: (1) fimH gene with its native signal was amplified from genomic J96 DNA with oligonucleotide primers GA13F and GA6R and (2) lac promoter/operator (lac p/o) from pPW19R was generated with primers GA11F and GA9R. Overlapping PCR using the primary fragments with primers GA11F and GA6R yielded a single fragment containing BglII and SalI.

Vector pCGA122-30 was made by a three part ligation encompassing (1) the BglII/SalI PCR fragment consisting of lac p/o, fimH native signal sequence+fimH; (2) the BglII/Sca1 fragment from pCGA101-8 containing the beta-lactamase gene, pUC ori, lac p/o, pelB leader, and fimC; and (3) the cassette containing kan^{r} and lacI^{q} from pTTQ18K as a SalI/Sca1 fragment.

Step 3

Replacement of pelB signal sequence 3′ to fimC with native signal sequence and removal of the amp^{r} gene to generate pCGA139-1-1.

The PelB signal 5′ to fimC was replaced with fimC native signal sequence, and cloned downstream of the first lac p/o utilizing overlapping PCR. Primary PCR fragments were generated as follows: (1) fimC with its native signal sequence was amplified from genomic J96 DNA with primers GA21F and GA2R; and (2) the lac p/o was amplified from pPW9R with primers GA24F and GA23R.

A single fragment containing fimC and its native signal preceded by the lac p/o resulted from overlapping PCR with primers ga24F and ga2R containing AflIII and BglII sites respectively. The product was cloned as a replacement AflIII/EcoR1 fragment into pCGA122-30 producing pCGA126-1.

The ampicillin resistance marker in pCGA126-1 was inactivated by interruption at the ScaI site in the beta-lactamase (bla) gene. This was followed by treatment with an exonuclease Bal31 and subsequent filling in with deoxynucleotide tri-phosphates (dNTP's). The plasmid was re-ligated resulting in a deletion of approximately 60 bases thus forming the plasmid pCGA139-1.

The parental pCGA126-1 vector was sequenced in its entirety. Specific sequences from regions of the pCGA126-1 plasmid including the fimC, fimH, kan^{r}, lacI^{q}, bla, and lac p/o. The deletion at the Sca1 site, giving rise to plasmid pCGA139-1, was confirmed by sequencing that locus in the derivative pCGA139-1 construct. Sensitivity to growth in the presence of ampicillin was also confirmed for E. coli containing the derivative pCGA139-1 vector. A single clone of pCGA139-1 was selected based on maximal expression of FimCH in the BL21 strain of E. coli. This clone was designated pCGA139-1-1 and was selected for clinical production of the FimCH vaccine.

The Host Cell Line

Plasmid pCGA139-1 was transformed into a BL21 E. coli host strain to optimize protein expression. Approximately twenty microliters of BL21 competent cells were pipetted into a pre-chilled 1.5 ml propylene tube. One microliter of vector was added and the mixture was incubated on ice for approximately 5 minutes. The tube was heat shocked in an approximately 42° C. water-bath for about 30 seconds followed by incubation on ice for approximately 2 minutes. SOC medium (approximately 80 ml) was added followed by incubation at 37° C. shaking at 250 rpm for approximately 1 hour. The culture was plated on 2XYT agar containing 50 μg/ml kanamycin and plates were incubated overnight at 37° C.

Plasmid preparations and frozen 15-20% glycerol stocks are made from individual colonies grown overnight in Terrific Broth (Quality Biologicals). Candidates are screened in replicate plates+/−ampicillin and individual colonies are chosen both by sensitivity to growth in the presence of 50 mg/ml ampicillin (for the replicate) and the absence of the Sca1 site in the bla gene. E. coli containing the plasmids are further analyzed for production of target protein. Six clones grown on 2XYT agar containing 50 mg/ml kanamycin are analyzed by restriction analysis pattern, Western blot analysis, and production of FimCH protein. 9 pCGA139-1-1 is selected as the final vector based on its yield of target protein. A single colony is grown overnight in Terrific Broth and aliquots stored in 15%-20% sterile glycerol in Nunc vials at −70° C.

Expression of the FimCH Construct

Overnight cultures are diluted 1:30 in Terrific Broth containing 50 mg/ml kanamycin and grown at 37° C. to mid-log phase (approximately 0.3 at OD600). Approximately 15 ml of each culture is induced with approximately 2.0 mM IPTG and harvested after approximately 3 hrs. Several 1 ml aliquots from each sample are sedimented in an eppendorf centrifuge at 14000 rpm for approximately 2 minutes. Total protein is estimated by BCA assay (Pierce) and 1.0 mg total protein of uninduced and induced culture is loaded onto two SDS-PAGE gels for electrophoresis and compared to FimCH standards of known concentration. Samples are also assayed using ion exchange chromatography for levels of FimCH protein.

Proteins are transferred to nitrocellulose membranes via Western blot, blocked with 2% dried milk and treated with primary polyclonal antibodies raised against FimC or truncated form of FimH expressed as a histidine-tagged fusion protein, FimH-T3. Membranes are washed three times (approximately 15 minutes each wash) with PBS plus 0.01% Tween-20 after which a donkey anti-rabbit secondary antibody conjugated to horseradish peroxidase (HRP) is applied for about 1 hour. Membranes are washed followed by treatment with an anti-HRP detection reagent, ECL, or ECL-plus. Nitrocellulose is finally exposed to x-ray films and developed in a M35A x-omatic processor.

Ion Exchange Chromatography of the FimCH Product

Samples were resuspended in approximately 200 ml of PBS , sonicated for approximately 12 minutes, and diluted 4-fold with PBS. Each sample was centrifuged at approximately 10,000 rpm for approximately 3 minutes into a 0.45 micron spin filter unit and transferred into HPLC microvials for analysis.

A Pharmacia Mono-S HR 5/5 column (5 mm×50 mm) was used for the quantification of Pilus proteins in analyzed samples. Mobile phase A was 20 mM potassium phosphate (pH 7.0); mobile phase B was mobile phase A containing 0.5 M potassium chloride. A gradient of 0%-30% B over 20 minutes was run at a flow rate of 1.25 ml/min. Eluted protein was detected using intrinsic tryptophan fluorescence detection (excitation 280 nm, emission 335 nm). A standard curve was generated using reference standard material diluted to concentrations from 5.2 μg/ml to 15.6 μg/ml. The correlation coefficient of the calibration curve was ³0.995.

The concentration of FimCH was determined using regression analysis from a standard curve of the area under the product peak. High levels of FimCH are typically seen in samples corresponding to pCGA139-1-1 in BL21 induced with IPTG. This clone was used because the high levels of FimCH expression seen in the IEC assay correlate with high expression that can be confirmed by Western blot analysis. The pCGA139-1-1 construct in BL21 corresponds to the construct used in the following examples and experiments. 6.2 Example 1

The immunogenicity of purified adhesin of strain J96 (having the amino acid sequence SEQ ID No.:4), adhesin-chaperone complex (using FimC from strain Nu14) (having an amino acid sequence of SEQ ID No.:2) and whole type 1 pili proteins were assessed by measuring immunoglobulin G (IgG) titer to FimHt adhesin (a naturally occurring FimH truncate corresponding to the NH₂-terminal two-thirds of the FimH protein (here, of strain J96) which was purified away from complexes of FimC and FimH (FimCH)) and whole type 1 pili, respectively, up to 78 weeks post immunization. Other FimH variant proteins, and their respective immunogenic truncates and fragments, are readily measured using the same protocol.

C3H/HeJ mice, five mice per group, were immunized on day 0 (primary immunization) (in Freund's adjuvant (CFA)) and booster immunization (week 4) (in incomplete Freund's adjuvant (IFA)) with one of the three antigens: purified truncated adhesin (FimHt), adhesin-chaperone complex (FimCH) or whole type 1 pili. Samples from individual mice treated identically were pooled for serological analysis and diluted 1:100 before serial dilution. Antibody responses were assessed by an ELISA with purified FimHt or whole pili as the capture antigens. Titers reflect the highest dilution of serum reacting twice as strongly as a comparable dilution of preimmune sera obtained from the same mice. The purity of the protein preparations of the capture antigens was 95% pure for whole type 1 pili and FimHt to 98 to 99% purity for FimC-H. In all cases the protein preparations were free of any lipopolysaccharide contaminants.

Both FimHt and FimCH induced strong, long-lasting immune responses to isolated FimHt and to FimH associated with whole type 1-pilus organelles. The responses persisted more than 30 weeks, and booster immunizations with FimHt or FimCH increased responsiveness. In contrast, type 1 pili elicited poor anti-FimH responses even though mice developed strong responses to whole pilus rods. Immunization studies in rabbits demonstrated similar immunogenicity profiles to those seen in mice. Antisera to FimHt and to FimCH bound to recombinant type 1+/FimH+E. coli strains (ORN103/pSH2) but not to the type 1+/FimH isogeneic mutant (ORN103/pUT2002) as determined by indirect immunofluorescence and flow cytometric analysis. Antibody to the whole pilus bound both ORN103/pSH2 and ORN103/pUT2002, as expected.

Comparable immune responses to the three antigens FimHt, FimCH and whole type 1 pili were seen in BALB/C and C57/BL6 strains of mice.

The role of FimH in adherence to cell surfaces, such as human bladder cells, has already been demonstrated, as has the efficacy of FimH-FimC complexes for use as immunogenic agents (U.S. patent application Ser. No. 09/298,494, filed Apr. 23, 1999, the disclosure of which is hereby incorporated by reference in its entirety).

### 6.3 Example 2

Passive immunization using the FimH variants of the present invention demonstrated as follows. Anti-sera against FimC and FimCH were generated and tested for reactivity with FimH variants. Two different pools were generated and used for these experiments. Mice were passively immunized intraperitoneally with 100 ml each of either anti-FimC or anti-FimCH rabbit sera 24 hours and 4 hours prior to inoculation. Endpoint titers for the sera were determined to be at least 1:500,000 by ELISA against the respective antigens.

Bacteria of different E. coli strains were then collected, washed and re-suspended in phosphate buffered saline (PBS) and cell concentration adjusted to OD=1.8 (at 600 nm). This suspension was then diluted 1:10 in PBS and tested for hemagglutination (HA) with guinea pig erythrocytes. This final suspension was used as inoculum and viability was determined on TSA plates. Mice were anaesthetized and then inoculated intraurethrally with 50 ml of E. coli suspension containing about 3×10⁷ colony forming units (CFU). Two days post-inoculation, the mice were sacrificed and bladders were removed and collected into 500 ml PBS supplemented with 1% mannose. The number of cfu's per bladder was determined by grinding the bilayers with a tissue tearer and then diluting and plating the suspension on TSA plates. The mean number of colony forming units per bladder was determined and data transformed to log CFU/bladder (as reported in Table 1).

TABLE 1
Passive Protection by FimH Variants

| | Mean Log CFU per Bladder | | | T-test | |
|---|---|---|---|---|---|
| Strain | FimC | FimCH | Naive | C vs. CH | CH vs. Naive |
| B223 | 7.79 | 5.69 | 7.58 | 0.0034 | 0.0107 |
| EC45 | 6.43 | 4.58 | ND | 0.0087 | ND |
| Nu14 | 4.54 | 2.53 | 5.22 | 0.0014 | 0.0000428 |
| B217 | 4.47 | 3.49 | 5.17 | 0.0142 | 0.0007 |
| DS17 | 4.64 | 3.02 | 4.45 | 0.0163 | 0.0355 |
| B218 | 4.30 | 2.99 | 4.16 | 0.0066 | 0.0331 |
| B220 | 4.18 | 1.93 | 3.55 | 0.0000257 | 0.0016 |
| EC56 | 3.02 | 2.60 | 3.34 | 0.5245 | 0.2222 |
| EC42 | 2.47 | 1.13 | 2.83 | 0.0274 | 0.0013 |
| J96 | 2.09 | 0.96 | 2.29 | 0.1005 | 0.0328 |
| B212 | 3.20 | 2.05 | 3.20 | 0.0167 | 0.443 |

### 6.4 Example 3

The purpose of this study was to examine the efficacy of FimCH to induce a protective immune response in primates.

A recombinant FimC and FimH complex was purified from E. coli K12 strain 600 extracted from the periplasm, and purified to over 99% purity as described in Jones et al. (PNAS 90:8397-401 (1993)).

Bacteria were cultivated in LB agar. Expression of type 1 pili was induced by two 48 hour passages in static brain-heart infusion broth (Difco Labs, Detroit) culture at 37° C. Before infection, expression of type 1 pili was quantitated by titration of bacterial suspension and mixing of equal volumes of 3% yeast cells and bacteria in microtiter cells. Bacterial suspensions showed agglutination titer of equal to or over 30-60. After bacterial challenge in the monkeys, urine samples from days 2, 4, 7 and 12 after challenge were counted by streaking 100 L of serial 10 step dilution onto cystine-lactose-electrolyte deficient agar plates by means of sterile plastic disposable loops. After incubation overnight at 37° C., E. coli colonies were counted to establish the number of cfu/ml in the urine. A urine specimen was considered positive when it contained at least 100 cfu/ml. To establish that inoculating strain was recovered in urine, urinary bacteria were biochemically analyzed on prepared microplates for rapid typing of coli form bacteria using PhenePlate systems.

FimH-T3, containing the amino terminal 163 of the 279 amino acids of FimH, was used in ELISAs. The surfactant stabilized emulsion adjuvant MF59 was used to emulsify the complex and for adjuvant administration. Cynomolgus monkeys received either 100 μg of FimCH in MF59 adjuvant at a 1:1 ratio, or MF59 plus diluent at weeks 0, 4, and 48. Each 1 ml injection was administered intramuscularly in the thigh, legs were alternated for each injection. There were four monkeys which received the vaccine composition and four control monkeys which received only the adjuvant.

Serum samples were collected once a month after vaccination for assessment of immune responses. The control monkeys did not have detectable anti-FimH antibodies in their serum at a 1:100 dilution of antiserum, which is the limit of detection of the assay, whereas the monkeys receiving the vaccine showed significant increase in anti-FimH titers upon the final booster at 48 weeks, ranging from an increase in 32 to 256 fold of anti-FimH titer.

Vaginal wash and serum samples were also collected before and after the last boost (weeks 47 and 50). The vaginal wash samples were diluted 1:2 in 0.5% bovine serum albumin, 0.5% milk and 0.2% azide before analysis. Antibody levels were recorded as actual OD at 405 nm; values <2x background were considered negative.

In addition, functional assays were performed with the serum and vaginal washes to demonstrated the efficacy of the vaccine to induce an anti-FimH immunoglobulin response.

With respect to the serum samples, type 1 piliated NU14 E. coli were directly labeled with fluorescein isothiocyanate and incubated with 106 J82 bladder cells at a ratio of 250 bacteria/cell in the presence of preimmune or immunized serum and incubated for 30 minutes at 37° C. After multiple washes, samples were assayed by flow cytometry, and percent inhibition was determined relative to preimmune samples from each monkey. Three of the four immunized monkeys serum resulted in almost 100% inhibition of NU 14 binding to the J82 cells and the fourth had approximately 90% inhibition relative to the non-immunized monkeys, which had either no or less than 20% inhibition of bacterial binding to the human bladder cells.

Vaginal washes were also tested to determine if the titer of antibodies in the washes of vaccinated subjects were sufficient to inhibit E. coli binding to trimannose. Briefly, 2.5 μg/ml of trimannose-bovine serum albumin was coated on Immulon-4 plates (Dynex Technologies, Chantilly, Va.). Type 1 piliated NU14 bacteria (8.0×10⁷ cfu/ml) was added to each well, incubated at 37° C. for one hour, washed extensively and bound bacteria were detected with 1:400 dilution of anti-E. coli horseradish peroxidase conjugated antibody (Biodesign, Kennebunk, Me.). Percent inhibition was assessed as a ratio, where % inhibition=[(full signal values−sample value)/full signal value]×100. Three of the four vaccinated monkeys demonstrated close to 100% inhibition of bacterial binding to the trimannose, whereas all four non-immunized monkeys showed less than 50% inhibition.

All eight test monkeys were infected 18 days after the final immunization with E. coli. Bladder infection was induced by inoculation of bacterial suspension (1 ml, 10⁸ cfu/ml) via urethral catheter. Urine samples were obtained on days 2, 4, 7, 12 and 14 after challenge to determine the number of bacteria per milliliter of urine, as a measure of infection. Urine samples were also tested for leukocytes as an indicator of inflammation. Three of the four immunized monkeys were completely protected from bladder infection and had no detectable bacteria or leukocytes in the urine on day 2 (limit of detection, 10² cfu/ml urine as in humans) and throughout the time period of the study.

Importantly, the immunized monkey that had serum anti-FimH antibodies, but did not have vaginal wash anti-FimH antibodies, was not protected from type 1 piliated bacteria challenge; additionally, none of the control monkeys were protected from challenge.

Normal flora was also tested to determine whether the vaccine affected E. coli growth. E. coli recovered from fecal suspensions from each monkey was tested in the PhP assay. All monkeys in both vaccine groups showed normal coliform bacterial growth. Thus, systemic vaccination with the FimH adhesin polypeptide does not appear to affect the normal intestinal flora.

These data clearly ,demonstrate that not only does a FimH derived vaccine composition induce an immune response in a primate sufficient to confer protection from bacterial UTI infection, but also that the protection is specifically derived from the presence of immunoglobulins secreted into the vaginal mucosal secretions.

### 6.5 Example 4

The purpose of this study was to examine the safety and immunogenicity of this FimCH composition formulated in the squalene-based adjuvant MF59C.1 in human subjects who were seronegative for anti-FimH antibodies. A FimCH composition used in the vaccine, i.e., FimCH is comprised of the FimC molecule, for example comprising the amino acid sequence depicted in SEQ ID No.:2 and FimC molecule, for example comprising the amino acid sequence depicted in SEQ ID No.:4, was tested in a randomized, controlled, double blind Phase I clinical trial in 48 healthy adult women.

Methods

The soluble 52 kDa recombinant protein complex of FimC and FimH, FimCH, was recovered from lysed bacteria using a three step chromatographic process. The bulk product is sterile filtered and vialed in a citrate buffer. Shortly before injection into a subject, the FimCH composition is mixed with a squalene-based emulsion adjuvant known as MF59C.1 (Chiron Corp., CA).

In vitro binding to human tissues, purified receptors or receptor homologues is often used to elucidate the roles in virulence of many different adhesins, including pilus-associated adhesins. Similarly, assaying for the ability of such antibodies to block attachment of bacteria to cells or specific receptors can assess the functionality of antibodies to adhesins. This allows for rapid in vitro assessment of serological cross-reactivity between antibodies raised to a single adhesin, such as FimCH purified from one strain of E. coli, against a wide range of E. Coli clinical isolates expressing highly homologous, yet phenotypically distinct FimH adhesins.

The ability of the anti-FimH adhesin antibodies to block bacterial binding to bladder epithelial cells is investigated in vitro using a flow cytometric method originally developed for evaluating Rickettsia-cell attachment (Li and Walker, Infect Immun., 60:2030-5, (1992), which is incorporated herein in its entirety).

The bacterial binding inhibition assay is run as follows. Type 1-piliated E. coli (cystitis, pyelonephritis, gut etc.) isolates are directly labeled with FITC and incubated with 2×10⁶ J82 bladder cells, at a ratio of 250 bacteria/cell, in the presence of pre-immune or hyper-immune serum (murine, rabbit, primate or human antisera) and allowed to mix with the bacteria for 30 minutes at 37° C. Antisera are added at dilutions typically ranging from 1:50 to 1:6400 (two-fold serial dilutions). After multiple washes, samples are assayed by flow cytometry in a FACStar PLUS (Becton Dickinson) according to previously published methods (Langermann et al., Science, 276:607-11(1997)). Mean channel fluorescence is used as an indicator of FITC-labeled bacteria bound to J82 bladder cells.

Endpoint inhibitory titers are defined as the titer, after serial two fold dilutions, at which the MCF value (representing bacteria bound to cells) is less than or equal to 50% of the MCF value for the control samples (where control is bacteria incubated with pre-immune serum). To confirm binding and inhibition, J82 bladder cells can be sorted from the flow cytometric adherence assay described and analyzed by fluorescent microscopy and the number of fluorescent bacteria attached to 40 bladder cells visually quantitated.

This assay can be run with vaginal wash samples as long as the samples are collected by straight lavage (“PBS washes”). For vaginal wash samples, inhibitory titer ratios are measured for all samples at a 1:2 dilution. Inhibition cannot be run with vaginal antibody samples collected by the cel-wec method, as this method relies upon a detergent-based extraction buffer which interferes with the binding assay.

Functional inhibitory antibodies to FimCH are also evaluated in an assay called the E. coli trimannose-binding assay. Briefly, Immulon-4 plates (Dynex Technologies, Inc., Chantilly, Va.) are coated with 2.5 μg/ml (100 ml/well) of tri-mannose-BSA (V-Labs, Covington, La.). Type 1-piliated NU14 (8.0×10⁷ cfu/ml) are added to each well, incubated at 37° C. for 1 hour and after extensive washing, bound bacteria are detected with a 1:400 dilution of an anti-E. coli-HRP conjugated antibody (Biodesign, Kennebunk, Me.). OD450 readings of these samples establish the full signal values (FSV) for binding to trimannose (approximately 2.0). Additional samples are run in the presence of 1:50 dilutions of serum to assess inhibition, where percent inhibition equals the FSV−the sample value/FSV×100. All samples are run in triplicate.

Antibody sampling of vaginal secretions from primates was performed with a sterile cotton swab. The swab was then suspended in 1 ml of PBS, yielding the solution to test for antibodies. The samples were centrifuged at 2,000×g for 10 minutes at 4° C. The supernatant was treated with Nonidet P-40, aliquoted and stored at −70° C. Antibody sampling of cervical secretions from humans was performed using an absorbent sponge called a Cel-Wec. Cervical secretions (Immunoglobulin) were eluted from sponges “Weck-Cel Spears” with elution buffer: 1×PBS, 0.5% Igepal® (nonionic detergent), Protease inhibitors (1 mg/ml Aprotinin, 1 mM Leupeptin, Bestatin). Antibody sampling of urine samples was done on straight, undiluted urine samples from “clean catch” specimens.

Quantitation of Human IgG in Serum/Urine/Cervical Secretion Samples ELISA Procedure

96 well ELISA plates are coated with capture antibody:

mouse anti human IgG (1 μg/ml CO3 buffer)

Standard*: Human IgG whole molecule (1000 ng-977 pg/ml)

Samples: Human urine or cervical secretions in PBS (diluted two fold 1:2 to 1:64)

Secondary: Biotin labeled goat F(ab′2) anti-human IgG

Tertiary: StrepAvidin Horse Radish Peroxidase

Substrate: TMB

Plates are read at 450 nm and quantity determined by Softmax software

* to generate a standard curve this is run along with the urine, cervical secretion samples

In order to determine IgG quantity, each urine and cervical secretion sample is run in duplicate at six different dilutions (for all individuals tested). The quantity for each dilution is automatically calculated by softmax using a 4 parameter standard curve (range 1000 ng-977 pg/ml). Only the quantities derived from OD values that fall within the linear range of the standard curve are used to determine the amount of IgG in a serum sample. These quantities are averaged to determine amount of IgG in a sample.

Clinical Results

Four cohorts of 12 subjects were randomized at a ratio of 3:1 (i.e., four groups where nine subjects received the vaccine and 3 subjects received the adjuvant alone) and, in a sequential fashion, given intramuscular doses of vaccine or control. FimCH was prepared for injection into a subject immediately prior to the injection, i.e., mixed with diluent and adjuvant. Doses of either 1, 5, 25 or 123 μg of FimCH in 0.5 mL of MF59C.1, or the control (MF59C.1 alone) were injected slowly, i.e., 20 to 30 seconds, into the deltoid muscle of the upper arm of the subjects at day 0, followed by a booster dose at about 28 days followed by a second booster dose at about 180 days.

The vaccine was safe and well tolerated at all doses upon administration of the vaccination protocol. Mild to moderate pain at the site of injection was the most common adverse event. In addition, mild or moderate headaches, fatigue, and myalgias were observed and all adverse events resolved within 3-4 days. No serious adverse events were reported and no subject was discontinued due to adverse events.

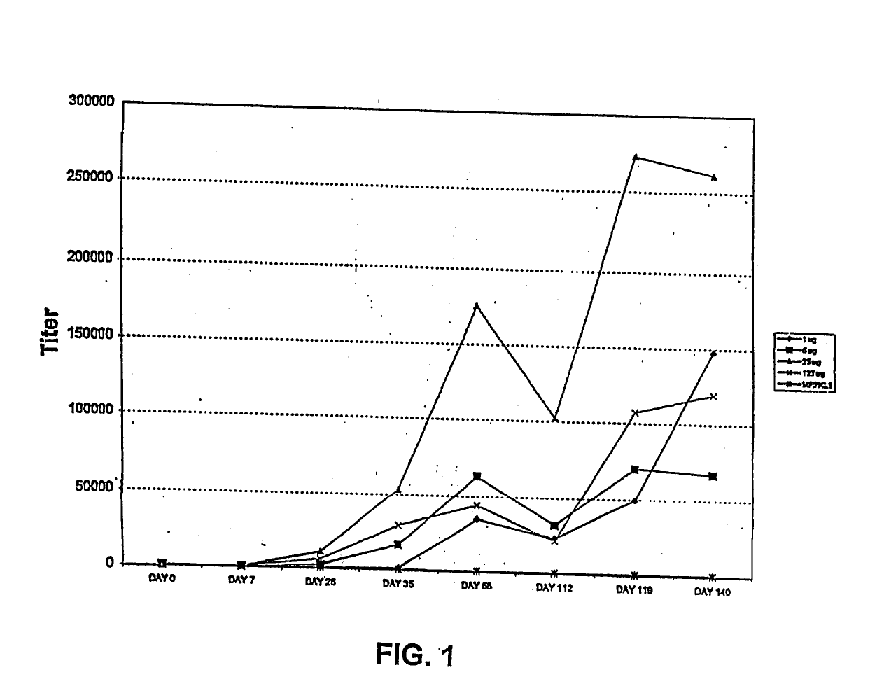

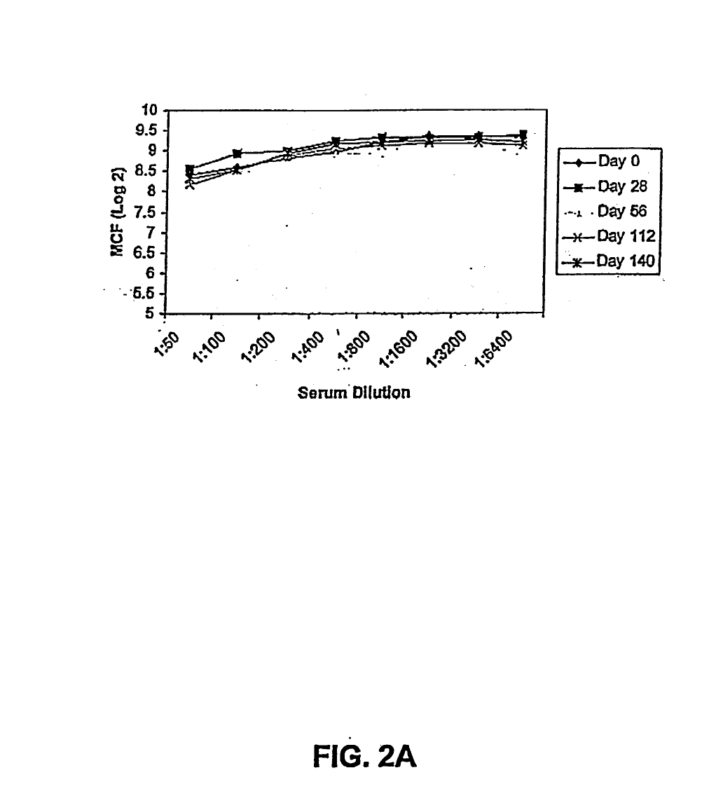

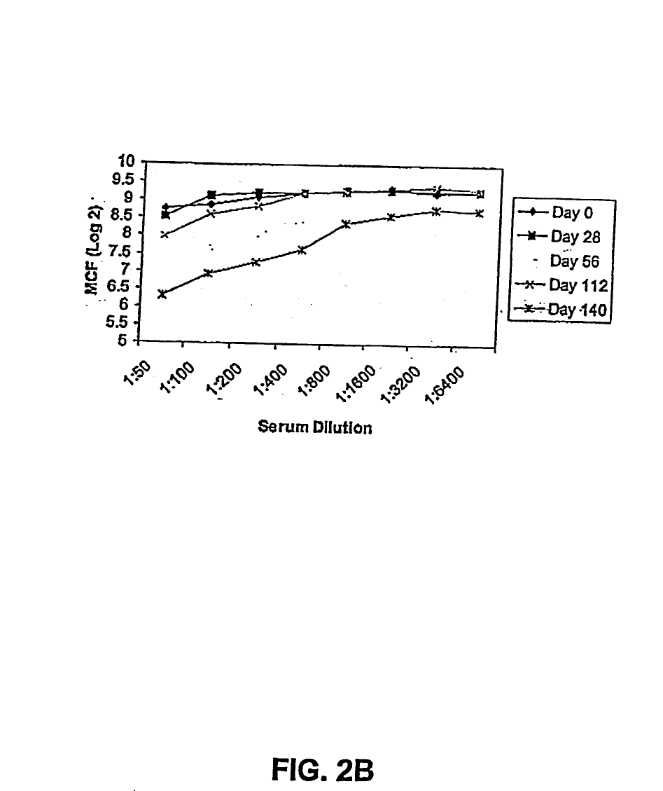

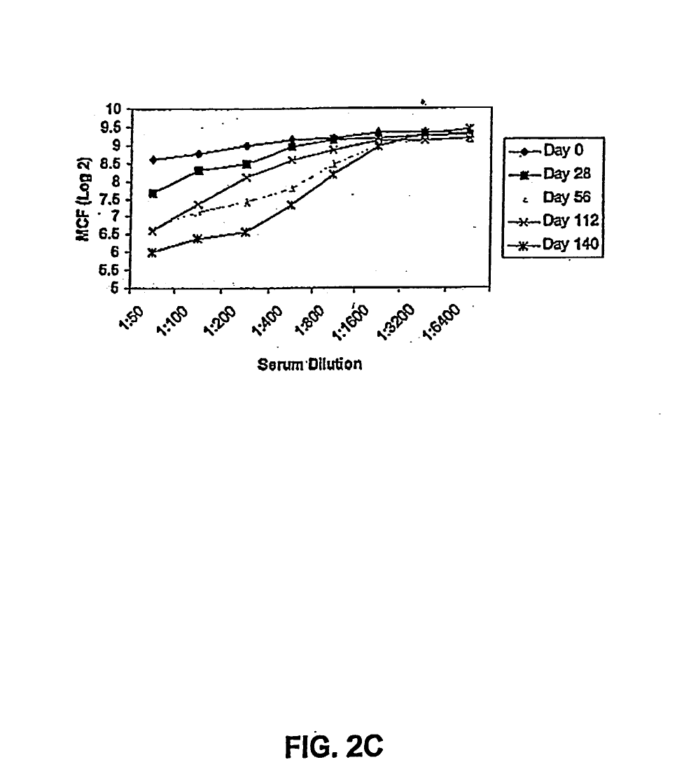

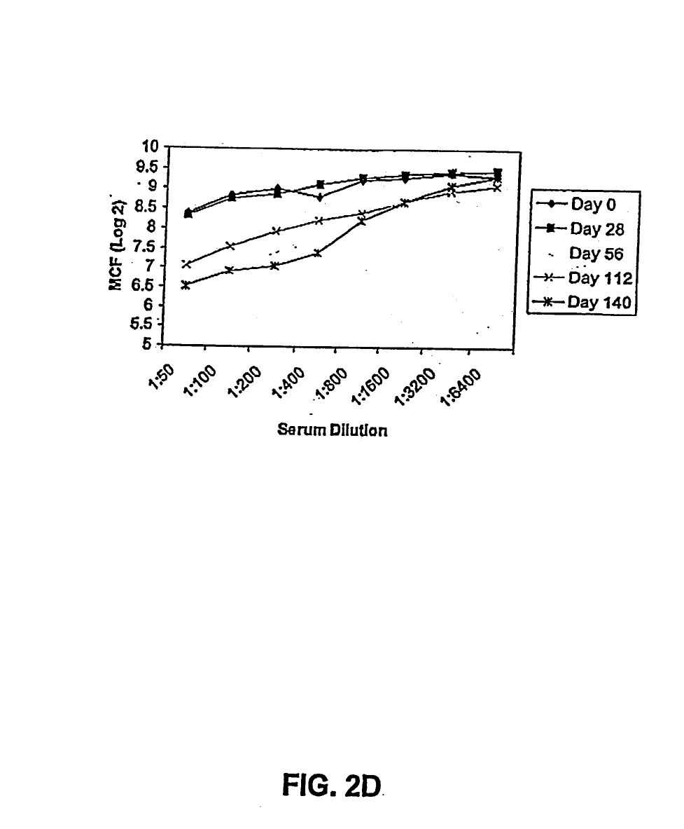

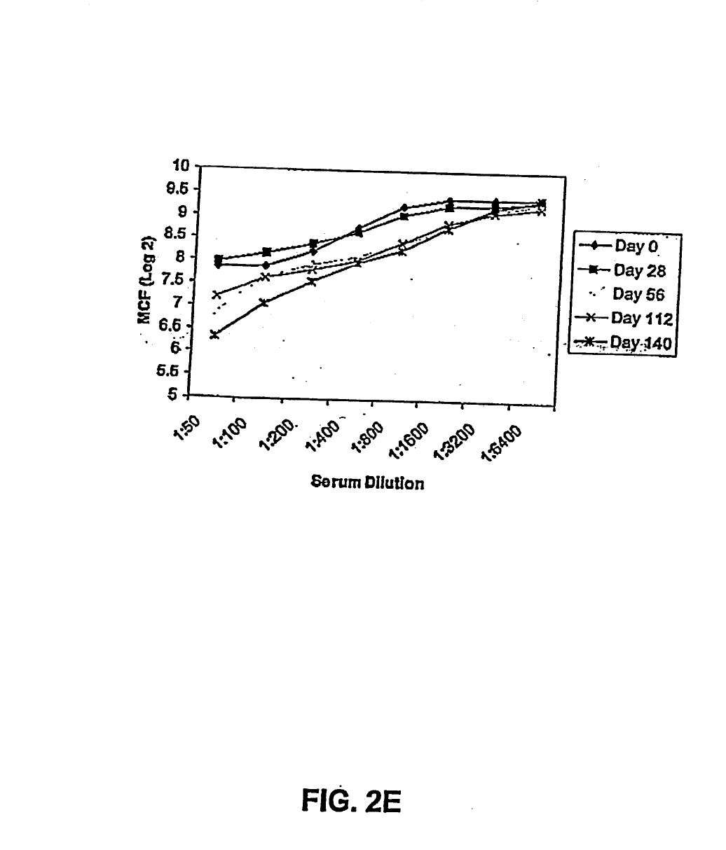

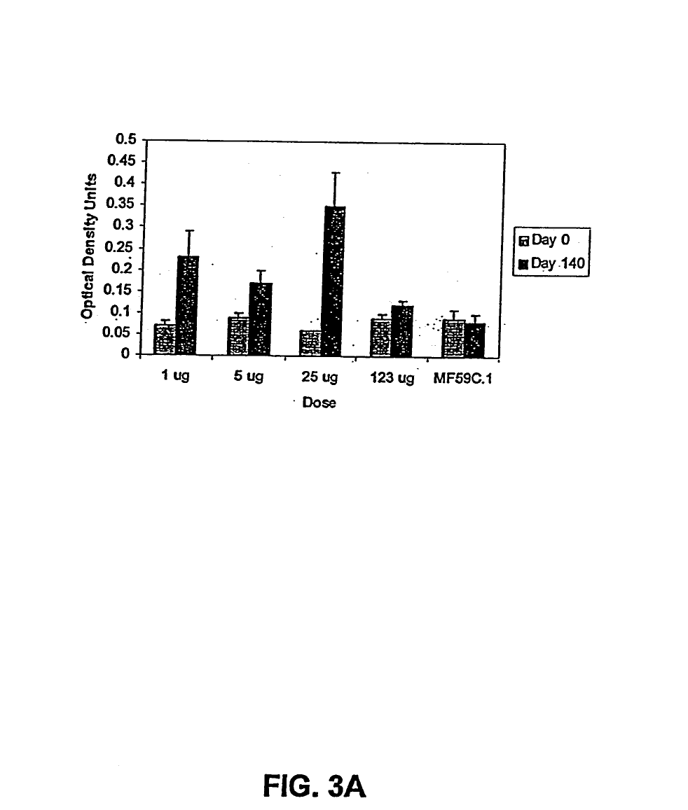

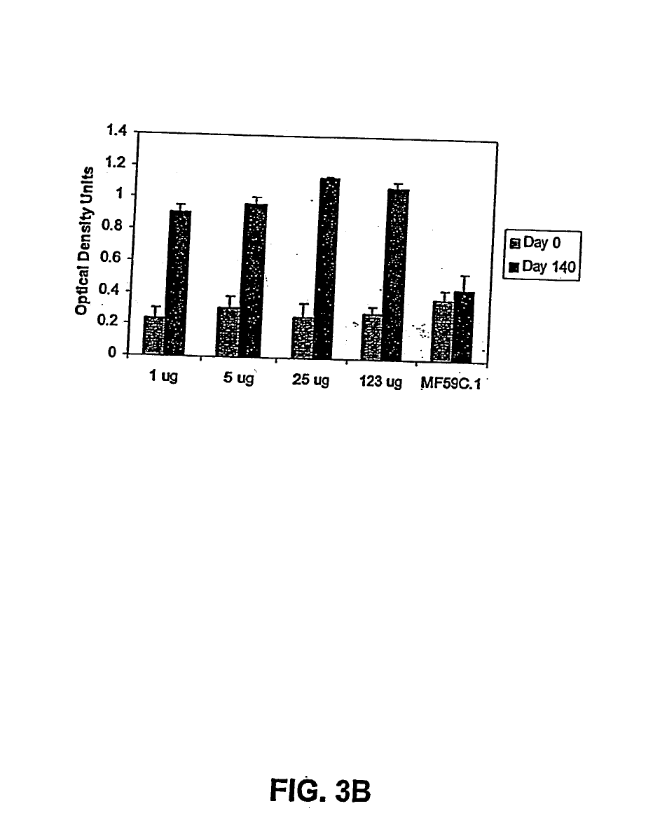

The FimCH vaccine was immunogenic in the human subjects and showed evidence of a clear dose response. All vaccine recipients developed serum IgG antibodies to FimH by ELISA (FIG. 1) and western blot. Subjects with the best serum responses, i.e., highest levels of anti-FimH-T3 IgGs, also had IgG against FimH detected in urine and vaginal secretions after immunization (FIG. 3A and FIG. 3B) and immune serum inhibited the binding of uropathogenic E. coli to a J82 human uroepithelial cell line (bladder cells) in vitro (FIGS. 2A-E).

All publications, patents and patent applications mentioned in this specification are herein incorporated by reference into the specification to the same extent as if each individual publication, patent or patent application was specifically and individually indicated to be incorporated herein by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of inducing in a primate immunoglobulin molecules that bind a polypeptide comprising an antigenic fragment of a type 1 adhesin that is associated with a bacterium causing urogenital tract infections, wherein said method comprises administering to a primate in need thereof a purified peptide or peptide complex comprising an antigenic fragment of a type 1 adhesin, in an amount effective to induce sufficient titers of said immunoglobulin molecules to reduce or prevent the incidence of urogenital tract infections in said primate.

2. The method of claim 1, wherein said immunoglobulin molecules are induced in the urine or genital secretions of the primate in sufficient titers to reduce or prevent the incidence of urogenital tract infections in said primate.

3. The method of claim 1, wherein said peptide or peptide complex corresponds to one or more β-sheet structures from an attachment domain of a type 1 adhesin.

4. The method of claim 1, wherein said peptide or peptide complex comprises an attachment domain of FimH or an antigenic fragment thereof.

5. The method of claim 1, wherein said peptide or peptide complex comprises at least 20 contiguous amino acids of FimH.

6. The method of claim 1 in which the purified peptide complex comprises a periplasmic chaperone protein, or fragment thereof.

7. The method of claim 1 in which the purified peptide complex is a FimCH complex.

8. The method of claim 1 in which the purified peptide complex contains equimolar amounts of a FimH protein having an amino acid sequence of SEQ ID No.:4 and a FimC protein having an amino acid sequence of SEQ ID No.:2.

9. The method of claim 1 in which the primate is a human.

10. The method of claim 8 in which the primate is a human.

11. The method of claim 2 in which the immunoglobulin molecules are IgG molecules.

12. The method of claim 1 in which the purified peptide or peptide complex is administered intravenously or intramuscularly.

13. The method of claim 1 in which the purified peptide or peptide complex is administered subcutaneously, transdermally, nasally or orally or by vaginal suppository.

14. The method of claim 1 or 2 in which the composition is not administered intraperitoneally.

15. The method of claim 10 in which approximately 1 μg of the purified peptide complex is administered.

16. The method of claim 10 in which approximately 25 μg of the purified peptide complex is administered.

17. The method of claim 10 in which between 20 μg and 30 μg of the purified peptide complex is administered.

18. The method of claim 1, wherein said method induces in the serum of said primate an endpoint titer of IgG molecules that specifically bind the type 1 adhesin of at least 3,200.

19. The method of claim 1, wherein said method induces in the serum of said primate a functional inhibitory ratio of at least 50% at a dilution of 1 to 50.

20. The method of claim 1 further comprising administering a second dose of said purified peptide or peptide complex approximately one month after a first administration.

21. The method of claim 1 or 20 further comprising administering a second or third dose of said purified peptide or peptide complex approximately six months after a first administration.

22. The method of claim 1 in which the purified peptide or peptide complex is administered in or with an adjuvant.

23. The method of claim 22 in which the adjuvant is squalene based.

24. The method of claim 1 in which the purified peptide or peptide complex is administered as a composition further comprising a citrate buffer.

25. The method of claim 24 in which said composition comprises 20 mM sodium citrate and 0.2 M NaCl, and has a pH of 6.0.

26. The method of claim 1 in which said urogenital tract infection is a urinary tract infection, a bladder infection or a kidney infection.

27. The method of claim 1 in which the disease is caused by a bacterium of the family Enterobacteriaceae.

28. The method of claim 1 in which the bacterium is E. coli.

29. The method of claim 10 in which said human suffered more than two urogenital infections within one year.

30. The method of claim 10 in which said human has asymptomatic bactourea.

31. The method of claim 30 in which said human is a pregnant woman or a diabetic.

32. The method of claim 10 in which said human is immunocompromised.

33. The method of claim 10 in which said human has an HIV infection, has cancer, or is in remission from cancer.

34. The method of claim 10 in which said human is at risk for end stage renal disease.

35. A method of inducing in a primate immunoglobulin molecules that inhibit binding of a bacterium causing urogenital tract infections to urogenital tract epithelial cells, wherein said method comprises administering to a primate in need thereof a purified peptide or peptide complex comprising an antigenic fragment of a type 1 adhesin, in an amount effective to induce sufficient titers of said immunoglobulin molecules in the urine or genital secretions of the primate to reduce or prevent the incidence of urogenital tract infections in said primate.

36. The method of claim 35, wherein said peptide or peptide complex corresponds to one or more β-sheet structures from an attachment domain of a type 1 adhesin.

37. The method of claim 35, wherein said peptide or peptide complex comprises an attachment domain of FimH or an antigenic fragment thereof.

38. The method of claim 35, wherein said peptide or peptide complex comprises at least 20 contiguous amino acids of FimH.

39. The method of claim 35 in which the purified peptide complex comprises a periplasmic chaperone protein, or fragment thereof.

40. The method of claim 35 in which the purified peptide complex is a FimCH complex.

41. The method of claim 35 in which the purified peptide complex contains equimolar amounts of a FimH protein having an amino acid sequence of SEQ ID No.:4 and a FimC protein having an amino acid sequence of SEQ ID No.:2.

42. The method of claim 35 in which the primate is a human.

43. The method of claim 41 in which the primate is a human.

44. The method of claim 35 in which the immunoglobulin molecules are IgG molecules.

45. The method of claim 35 in which the purified peptide or peptide complex is administered intravenously or intramuscularly.

46. The method of claim 35 in which the purified peptide or peptide complex is administered subcutaneously, transdermally, nasally or orally or by vaginal suppository.

47. The method of claim 35 in which the composition is not administered intraperitoneally.

48. The method of claim 43 in which approximately 1 μg of the purified peptide complex is administered.

49. The method of claim 43 in which approximately 25 μg of the purified peptide complex is administered.

50. The method of claim 43 in which between 20 μg and 30 μg of the purified peptide complex is administered.

51. The method of claim 35 which also induces in the serum of said primate an endpoint titer of IgG molecules that specifically bind the type 1 adhesin of at least 3,200.

52. The method of claim 35 which also induces in the serum of said primate of a functional inhibitory ratio of at least 50% at a dilution of 1 to 50.

53. The method of claim 35 further comprising administering a second dose of said purified peptide or peptide complex approximately one month after a first administration.

54. The method of claim 35 or 53 further comprising administering a second or third dose of said purified peptide or peptide complex approximately six months after a first administration.

55. The method of claim 35 in which the purified peptide or peptide complex is administered in or with an adjuvant.

56. The method of claim 55 in which the adjuvant is squalene based.

57. The method of claim 35 in which the purified peptide or peptide complex is administered as a composition further comprising a citrate buffer.

58. The method of claim 57 in which said composition comprises 20 mM sodium citrate and 0.2 M NaCl, and has a pH of 6.0.

59. The method of claim 35 in which said urogenital tract infection is a urinary tract infection, a bladder infection or a kidney infection.

60. The method of claim 35 in which the disease is caused by a bacterium of the family Enterobacteriaceae.

61. The method of claim 35 in which the bacterium is E. coli.

62. The method of claim 43 in which said human suffered more than two urogenital infections within one year.

63. The method of claim 43 in which said human has asymptomatic bactourea.

64. The method of claim 63 in which said human is a pregnant woman or a diabetic.

65. The method of claim 43 in which said human is immunocompromised.

66. The method of claim 43 in which said human has an HIV infection, has cancer, or is in remission from cancer.

67. The method of claim 43 in which said human is at risk for end stage renal disease.

68. A method for vaccinating a primate against urogenital tract infection, wherein said method comprises administering to a primate a purified peptide or peptide complex comprising an antigenic fragment of a type 1 adhesin, in an amount effective to induce titers of immunoglobulin that reduce or prevent the incidence of urogenital tract infections in said primate.

69. The method of claim 68, wherein said peptide or peptide complex corresponds to one or more β-sheet structures from an attachment domain of a type 1 adhesin.

70. The method of claim 68, wherein said peptide or peptide complex comprises an attachment domain of FimH or an antigenic fragment thereof.

71. The method of claim 68, wherein said peptide or peptide complex comprises at least 20 contiguous amino acids from FimH.

72. The method of claim 68 in which said immunoglobulin molecules are present in the serum of the primate.

73. The method of claim 68 in which said immunoglobulin molecules are present in the urine or genital tract secretions of the primate.

74. The method of claim 73 in which said immunoglobulin molecules are present at a level sufficient to reduce the incidence of the urogenital tract infection.

75. The method of claim 73 in which said immunoglobulin molecules inhibit binding of said bacterium to urogenital tract epithelial cells.

76. The method of claim 73 in which said immunoglobulin molecules are also present in the serum of the primate

77. The method of claim 68 in which the purified peptide complex comprises a periplasmic chaperone protein, or fragment thereof.

78. The method of claim 68 in which the purified peptide complex is a FimCH complex.

79. The method of claim 68 in which the purified peptide complex contains equimolar amounts of a FimH protein having an amino acid sequence of SEQ ID No.:4 and a FimC protein having an amino acid sequence of SEQ ID No.:2.

80. The method of claim 68 in which the primate is a human.

81. The method of claim 79 in which the primate is a human.

82. The method of claim 73 in which the immunoglobulin molecules are IgG molecules.

83. The method of claim 68 in which the purified peptide or peptide complex is administered intravenously or intramuscularly.

84. The method of claim 68 in which the purified peptide or peptide complex is administered subcutaneously, transdermally, nasally or orally or by vaginal suppository.

85. The method of claim 73 in which the composition is not administered intraperitoneally.

86. The method of claim 81 in which approximately 1 μg of the purified peptide complex is administered.

87. The method of claim 81 in which approximately 25 μg of the purified peptide complex is administered.

88. The method of claim 81 in which between 20 μg and 30 μg of the purified peptide complex is administered.

89. The method of claim 68 further comprising administering a second dose of said purified peptide or peptide complex approximately one month after a first administration.

90. The method of claim 68 or 89 further comprising administering a second or third dose of said purified peptide or peptide complex approximately six months after a first administration.

91. The method of claim 68 in which the purified peptide or peptide complex is administered in or with an adjuvant.

92. The method of claim 91 in which the adjuvant is squalene based.

93. The method of claim 68 in which the purified peptide or peptide complex is administered as a composition further comprising a citrate buffer.

94. The method of claim 93 in which said composition comprises 20 mM sodium citrate and 0.2 M NaCl, and has a pH of 6.0.

95. The method of claim 68 in which said urogenital tract infection is a urinary tract infection, a bladder infection or a kidney infection.

96. The method of claim 68 in which the urinary tract infection is caused by a bacterium of the family Enterobacteriaceae.

97. The method of claim 68 in which the bacterium is E. coli.

98. The method of claim 81 in which said human suffered more than two urogenital infections within one year.

99. The method of claim 81 in which said human has asymptomatic bactourea.

100. The method of claim 99 in which said human is a pregnant woman or a diabetic.

101. The method of claim 81 in which said human is immunocompromised.

102. The method of claim 81 in which said human has an HIV infection, has cancer, or is in remission from cancer.

103. The method of claim 81 in which said human is at risk for end stage renal disease.

104. A method for slowing or preventing, in a primate in need thereof, progression of a urinary tract infection into end stage renal disease, wherein said method comprises administering to a primate a purified peptide or peptide complex comprising an antigenic fragment of a type 1 adhesin, in an amount effective to induce titers of immunoglobulin that slow or prevent progression of a urinary tract infection into end stage renal disease.

105. The method of claim 104, wherein said peptide or peptide complex corresponds to one or more β-sheet structures from an attachment domain of a type 1 adhesin.

106. The method of claim 104, wherein said peptide or peptide complex comprises an attachment domain of FimH or antigenic fragments thereof.

107. The method of claim 104, wherein said peptide or peptide complex comprises at least 20 contiguous amino acids of FimH.

108. The method of claim 104 in which said immunoglobulin molecules are present in the serum of the primate.

109. The method of claim 104 in which said immunoglobulin molecules are present in the urine or genital tract secretions of the primate.

110. The method of claim 109 in which said immunoglobulin molecules are present at a level sufficient to prevent or reduce the progression into end stage renal disease.

111. The method of claim 109 in which said immunoglobulin molecules inhibit binding of said bacterium to urogenital tract epithelial cells.

112. The method of claim 109 in which said immunoglobulin molecules are also present in the serum of the primate

113. The method of claim 104 in which the purified peptide complex comprises a periplasmic chaperone protein, or fragment thereof.

114. The method of claim 104 in which the purified peptide complex is a FimCH complex.

115. The method of claim 114 in which the purified peptide complex contains equimolar amounts of a FimH protein having an amino acid sequence of SEQ ID No.:4 and a FimC protein having an amino acid sequence of SEQ ID No.:2.

116. The method of claim 104 in which the primate is a human.

117. The method of claim 115 in which the primate is a human.

118. The method of claim 109 in which the immunoglobulin molecules are IgG molecules.

119. The method of claim 104 in which the purified peptide or peptide complex is administered intravenously or intramuscularly.

120. The method of claim 104 in which the purified peptide or peptide complex is administered subcutaneously, transdermally, nasally or orally or by vaginal suppository.

121. The method of claim 109 in which the composition is not administered intraperitoneally.

122. The method of claim 117 in which approximately 1 μg of the purified peptide complex is administered.

123. The method of claim 117 in which approximately 25 μg of the purified peptide complex is administered.

124. The method of claim 117 in which between 20 μg and 30 μg of the purified peptide complex is administered.

125. The method of claim 104 further comprising administering a second dose of said purified peptide or peptide complex approximately one month after a first administration.

126. The method of claim 104 or 117 further comprising administering a second or third dose of said purified peptide or peptide complex approximately six months after a first administration.

127. The method of claim 104 in which the purified peptide or peptide complex is administered in or with an adjuvant.

128. The method of claim 127 in which the adjuvant is squalene based.

129. The method of claim 104 in which the purified peptide or peptide complex is administered as a composition further comprising a citrate buffer.

130. The method of claim 129 in which said composition comprises 20 mM sodium citrate and 0.2 M NaCl, and has a pH of 6.0.

131. The method of claim 104 in which the urinary tract infection is caused by a bacterium of the family Enterobacteriaceae.

132. The method of claim 104 in which the bacterium is E. coli.

133. The method of claim 117 in which said human has asymptomatic bactourea.

134. The method of claim 133 in which said human is a pregnant woman or a diabetic.

135. A method for treating or ameliorating, in a primate in need thereof, the symptoms of a urogenital tract infection, wherein said method comprises administering to a primate a purified peptide or peptide complex comprising an antigenic fragment of a type 1 adhesin, in an amount effective to induce titers of immunoglobulin that treat or ameliorate the symptoms of a urogenital tract infection.

136. The method of claim 135, wherein said peptide or peptide complex corresponds to one or more β-sheet structures from an attachment domain of a type 1 adhesin.

137. The method of claim 135, wherein said peptide or peptide complex comprises an attachment domain of FimH or antigenic fragments thereof.

138. The method of claim 135, wherein said peptide or peptide complex comprises at least 20 contiguous amino acids from FimH.

139. The method of claim 135 in which said immunoglobulin molecules are present in the serum of the primate.

140. The method of claim 135 in which said immunoglobulin molecules are present in the urine or genital tract secretions of the primate.

141. The method of claim 140 in which said immunoglobulin molecules are present at a level sufficient to treat or ameliorate the symptoms of the urogenital tract infection.

142. The method of claim 135 in which said immunoglobulin molecules inhibit binding of said bacterium to urogenital tract epithelial cells.

143. The method of claim 140 in which said immunoglobulin molecules are also present in the serum of the primate

144. The method of claim 135 in which the purified peptide complex comprises a periplasmic chaperone protein, or fragment thereof.

145. The method of claim 144 in which the purified peptide complex is a FimCH complex.

146. The method of claim 135 in which the purified peptide complex contains equimolar amounts of a FimH protein having an amino acid sequence of SEQ ID No.:4 and a FimC protein having an amino acid sequence of SEQ ID No.:2.

147. The method of claim 135 in which the primate is a human.

148. The method of claim 146 in which the primate is a human.

149. The method of claim 141 in which the immunoglobulin molecules are IgG molecules.

150. The method of claim 135 in which the purified peptide or peptide complex is administered intravenously or intramuscularly.

151. The method of claim 135 in which the purified peptide or peptide complex is administered subcutaneously, transdermally, nasally or orally or by vaginal suppository.

152. The method of claim 141 in which the composition is not administered intraperitoneally.

153. The method of claim 148 in which approximately 1 μg of the purified peptide complex is administered.

154. The method of claim 148 in which approximately 25 μg of the purified peptide complex is administered.

155. The method of claim 135 in which the purified peptide or peptide complex is administered in or with an adjuvant.

156. The method of claim 155 in which the adjuvant is squalene based.

157. The method of claim 135 in which said urogenital tract infection is a urinary tract infection, a bladder infection or a kidney infection.

158. The method of claim 135 in which the urinary tract infection is caused by a bacterium of the family Enterobacteriaceae.

159. The method of claim 135 in which the bacterium is E. coli.

160. The method of claim 148 in which said human has asymptomatic bactourea.

161. The method of claim 160 in which said human is a pregnant woman or a diabetic.

162. A method for vaccinating a primate against urogenital tract infection, which method comprises administering to the primate a purified nucleic acid containing a nucleotide sequence encoding a peptide or peptide complex comprising a an antigenic fragment of a type 1 pilin polypeptide associated with a bacterium that causes a urogenital tract infection, said purified nucleic acid being administered in an amount effective to produce immunoglobulin molecules that specifically bind the type 1 pilin.

163. The method of claim 162 in which said immunoglobulin molecules are present in the serum of the primate.

164. The method of claim 162 or 163 in which said immunoglobulin molecules are present in the urine or genital tract secretions of the primate.

165. The method of claim 162 in which said immunoglobulin molecules are present at a level sufficient to reduce the incidence of the urogenital tract infection.

166. The method of claim 162 in which said immunoglobulin molecules inhibit binding of said bacterium to urogenital tract epithelial cells.

167. The method of claim 162 in which said peptide or peptide complex comprises an attachment domain of FimH.

168. The method of claim 162 in which the peptide complex comprises a periplasmic chaperone protein, or fragment thereof.

169. The method of claim 168 in which the peptide complex is a FimCH complex.

170. The method of claim 162 in which the peptide complex contains a FimH protein having an amino acid sequence of SEQ ID No.:4 and a FimC protein having an amino acid sequence of SEQ ID No.:2.

171. A method of inducing immunoglobulin molecules, that specifically bind an attachment domain of a type 1 pilin polypeptide associated with a bacterium that causes urogenital tract infections, in the urine or genital tract secretions of a primate, which method comprises administering to a primate in need thereof a purified peptide or peptide complex comprising said type 1 pilin attachment domain, in an amount effective to induce a level of said immunoglobulin molecules in the serum of said primate sufficient to reduce the incidence of urogenital tract infections.

172. A method of inducing immunoglobulin molecules that inhibit binding of a bacterium, which bacterium causes urogenital tract infections, to urogenital tract epithelial cells, in the urine or genital tract secretions of a primate, which method comprises administering to a primate in need thereof a purified peptide or peptide complex comprising an attachment domain of a type 1 pilin polypeptide associated with the bacterium, in an amount effective to induce a level of said immunoglobulin molecules in the serum of said primate sufficient to reduce the incidence of urogenital tract infections.

173. A pharmaceutical composition comprising a purified peptide complex of a FimH protein having an amino acid sequence of SEQ ID No.:4 and a FimC protein having an amino acid sequence of SEQ ID No.:2, said pharmaceutical composition being suitable for administration to humans.

174. The pharmaceutical composition of claim 173 which further comprises a carrier.

175. The pharmaceutical composition of claim 173 which is in a solid form.

176. The pharmaceutical composition of claim 173 which is lyophilized.

177. The pharmaceutical composition of claim 173 which is in a liquid form.

178. The pharmaceutical composition of claim 177 which comprises a sterile isotonic aqueous buffer.

179. The pharmaceutical composition of claim 178 in which said sterile isotonic aqueous buffer is a citrate buffer.

180. The pharmaceutical composition of claim 179 which comprises 20 mM sodium citrate and 0.2 M NaCl, and has a pH of 6.0.

181. The pharmaceutical composition of claim 173 which further comprises an adjuvant.

182. The pharmaceutical composition of claim 181 in which the adjuvant is squalene based.

183. The pharmaceutical composition of claim 173 in which said composition is non-pyrogenic.

184. A thermally stable pharmaceutical composition that is suitable for reconstitution into an injectable sterile and particulate-free solution which comprises a purified peptide complex of a FimH protein having the amino acid sequence of SEQ ID No.:4 and a FimC protein having the amino acid sequence of SEQ ID. No.:2.

185. A chemically stable pharmaceutical composition that is suitable for reconstitution into an injectable sterile and particulate-free solution which comprises a purified peptide complex of a FimH protein having the amino acid sequence of SEQ ID No.:4 and a FimC protein having the amino acid sequence of SEQ ID No.:2.

186. A sterile unit dosage form comprising 490 μg/ml of a purified peptide complex of a FimH protein having the amino acid sequence of SEQ ID No.:4 and a FimC protein having the amino acid sequence of SEQ ID No.:2.

187. The sterile unit dosage form of claim 186 which is in a sealed container.

188. A kit comprising a first container comprising a first composition comprising of a purified peptide complex of a FimH protein having the amino acid sequence of SEQ ID No.:4 and a FimC protein having the amino acid sequence of SEQ ID No.:2 and a second container comprising a second composition comprising an adjuvant, wherein both said first and second compositions are suitable for administration to a human.

189. The kit of claim 188 in which the first composition further comprises a carrier.

190. The kit of claim 188 in which the first composition is in a solid form.

191. The kit of claim 190 in which the first composition is lyophilized.

192. The kit of claim 188 in which the first composition is in a liquid form.

193. The kit of claim 192 in which the first composition further comprises a sterile isotonic aqueous buffer.

194. The kit of claim 193 in which said sterile isotonic aqueous buffer is a citrate buffer.

195. The kit of claim 194 in which said first composition comprises 20 mM sodium citrate and 0.2 M NaCl, and has a pH of 6.0.

196. The kit of claim 188 in which said first and second compositions are non-pyrogenic.

197. The kit of claim 188 in which the adjuvant is squalene based.

198. A pharmaceutical formulation comprising a purified peptide or peptide complex that comprises an antigenic fragment of a type 1 adhesin associated with a bacterium causing urogenital tract infections, wherein said pharmaceutical formulation contains an appropriate dose of said peptide or peptide complex to induce, when administered to a primate, immunoglobulin titers sufficient to reduce or prevent the incidence of urogenital tract infections in said primate.

199. The pharmaceutical formulation of claim 198, wherein said purified peptide or peptide complex comprises the attachment domain of FimH or antigenic fragments thereof.

200. The pharmaceutical formulation of claim 199, wherein said purified peptide or peptide complex corresponds to one or more β-sheet structures from the attachment domain of FimH.

201. The pharmaceutical formulation of claim 200, wherein said antigenic fragments comprise at least 20 contiguous amino acids from FimH.

202. The pharmaceutical formulation of claim 200, wherein said formulation includes a squalene-based emulsion adjuvant.

203. The pharmaceutical formulation of claim 200, wherein said purified peptide complex comprises a FimCH complex.

204. The pharmaceutical formulation of claim 200, wherein said appropriate dose is within the range from about 1 μg to about 200 μg of peptide or peptide complex.

205. The pharmaceutical formulation of claim 204, wherein said appropriate dose is within the range from about 1 μg to about 200 μg of FimCH complex.

206. The pharmaceutical formulation of claim 204, wherein said appropriate dose is within the range from about 1 μg to about 20 μg of FimCH complex.

207. The pharmaceutical formulation of claim 204, wherein said appropriate dose is within the range from about 20 μg to about 30 μg of FimCH complex.

208. The pharmaceutical formulation of claim 204, wherein said appropriate dose is within the range from about 30 μg to about 50 μg of FimCH complex.

209. The pharmaceutical formulation of claim 204, wherein said appropriate dose is selected from the group consisting of 1, 5, 20, 25, 30, 50, 100, 123 and 200 μg of FimCH complex.
